(19) United States
(12) Patent Application Publication
(10) Pub. No.: US 2025/0019444 A1
(43) Pub. Date: Jan. 16, 2025

(54) ANTIBODY VARIABLE DOMAINS TARGETING THE NKG2D RECEPTOR

(71) Applicant: Dragonfly Therapeutics, Inc., Waltham, MA (US)

(72) Inventors: Gregory P. Chang, Medford, MA (US); Ann F. Cheung, Lincoln, MA (US); Asya Grinberg, Lexington, MA (US); William Haney, Wayland, MA (US); Bradley M. Lunde, Lebanon, NH (US); Bianka Prinz, Lebanon, NH (US)

(21) Appl. No.: 18/894,660

(22) Filed: Sep. 24, 2024

Related U.S. Application Data
(62) Division of application No. 18/501,419, filed on Nov. 3, 2023, now Pat. No. 12,129,300.
Application No. 18/501,419 is a division of application No. 16/967,216, filed on Aug. 4, 2020, now Pat. No. 11,884,733.
(60) Provisional application No. 62/716,259, filed on Aug. 8, 2018.
(60) Provisional application No. 62/628,161, filed on Feb. 8, 2018.

(51) Int. Cl. C07K 16/28; C07K 16/32

(52) U.S. Cl. CPC C07K 16/2851; C07K 16/2803; C07K 16/283; C07K 16/32; C07K 2317/31; C07K 2317/52; C07K 2317/56; C07K 2317/565; C07K 2317/73; C07K 2317/92; C07K 2317/94

(57) ABSTRACT

Antibody heavy chain variable domains that can be paired with antibody light chain variable domains to form an antigen-binding site targeting the NKG2D receptor on natural killer cells are described. Proteins comprising an NKG2D antigen-binding site, pharmaceutical compositions and therapeutic methods thereof, including for the treatment of cancer, are also described.

### CROSS-REFERENCE TO RELATED APPLICATIONS

This application is a divisional of U.S. patent application Ser. No. 18/501,419, filed Nov. 3, 2023, which is a divisional of U.S. patent application Ser. No. 16/967,216, filed on Aug. 4, 2020, which is a U.S. national stage application, filed under 35 U.S.C. § 371, of International Application No. PCT/US2019/017330, filed on Feb. 8, 2019, which claims the benefit of and priority to U.S. Provisional Patent Application No. 62/628,161, filed on Feb. 8, 2018, and U.S. Provisional Patent Application No. 62/716,259, filed on Aug. 8, 2018. The disclosure of each of the applications above, except for U.S. Provisional Patent Application No. 62/716,259, is incorporated by reference herein in its entirety for all purposes.

### SEQUENCE LISTING

The instant application contains a Sequence Listing which has been submitted electronically in XML format and is hereby incorporated by reference in its entirety. Said XML copy, created on Sep. 18, 2023, is named DFY-048D4.xml and is 114,286 bytes in size.

### FIELD OF THE INVENTION

The invention provides proteins with antibody heavy chain and light chain variable domains that can be paired to form an antigen-binding site targeting the Natural Killer group 2D (NKG2D) receptor on natural killer cells, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, for example WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for priming, which distinguishes them from T cells. Activated NK cells kill target cells by means similar to cytotoxic T cells—i.e., via cytolytic granules that contain perforin and granzymes as well as via death receptor pathways. Activated NK cells also secrete inflammatory cytokines such as IFN-gamma and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals. NKG2D is a type-II transmembrane protein that is expressed by essentially all natural killer cells where NKG2D serves as an activating receptor. The ability to modulate NK cell function via NKG2D is useful in various therapeutic contexts including malignancy.

### SUMMARY

Antibodies to NKG2D have been identified that provide important advantages in the design of therapeutic agents. For example, some of these antibodies do not merely bind human NKG2D receptor, but have one or more further advantages such as the ability to agonize the receptor; the ability to compete with a natural ligand for binding to the receptor; and/or the ability to cross-react with NKG2D from other species such as cynomolgus monkey. These advantages can be achieved across a range of affinities for NKG2D.

Accordingly, one aspect of the invention relates to an antibody heavy chain variable domain at least 90% identical to the amino acid sequence QVQLVQSGAEVKKPGASVKVSCKASGYTFTSYYMHWVRQAPGQGLEWMGIINPSGGS TSYAQKFQGRVTMTRDTSTSTVYMELSSLRSEDTAVYYCARGAPNYGDTTHDYYYMD VWGKGTTVTVSS (SEQ ID NO:1, ADI-29379). In some embodiments, the antibody heavy chain variable domain is at least 95% identical to SEQ ID NO:1. In some embodiments, the heavy chain variable domain includes amino acid sequences YTFTSYYMH (SEQ ID NO:11) as the first complementarity-determining region 1 (“CDR1”), IINPSGGSTSYAQKFQG (SEQ ID NO: 12) as the second CDR (“CDR2”), and ARGAPNYGDTTHDYYYMDV (SEQ ID NO:13) as the third CDR (“CDR3”) of SEQ ID NO:1. In some embodiments, the heavy chain variable domain includes amino acid sequences SYYMH (SEQ ID NO:45) as CDR1, IINPSGGSTSYAQKFQG (SEQ ID NO:12) as CDR2, and GAPNYGDTTHDYYYMDV (SEQ ID NO: 68) as CDR3 of SEQ ID NO:1.

Another aspect of the invention relates to an antibody heavy chain variable domain at least 90% identical to the amino acid sequence QVQLVQSGAEVKKPGASVKVSCKASGYTFTGYYMHWVRQAPGQGLEWMGWINPNSG GTNYAQKFQGRVTMTRDTSISTAYMELSRLRSDDTAVYYCARDTGEYYDTDDHGMDV WGQGTTVTVSS (SEQ ID NO: 3, ADI-29463). In some embodiments, the antibody heavy chain variable domain is at least 95% identical to SEQ ID NO:3. In some embodiments, the heavy chain variable domain includes amino acid sequences YTFTGYYMH (SEQ ID NO: 17) as the first complementarity-determining region (“CDR1”), WINPNSGGTNYAQKFQG (SEQ ID NO: 18) as the second CDR (“CDR2”), and ARDTGEYYDTDDHGMDV (SEQ ID NO:19) as the third CDR (“CDR3”) of SEQ ID NO:3. In some embodiments, the heavy chain variable domain includes amino acid sequences GYYMH (SEQ ID NO: 92) as CDR1, WINPNSGGTNYAQKFQG (SEQ ID NO:18) as CDR2, and DTGEYYDTDDHGMDV (SEQ ID NO: 69) as CDR3 of SEQ ID NO:3.

Another aspect of the invention relates to an antibody heavy chain variable domain at least 90% identical to the amino acid sequence EVQLLESGGGLVQPGGSLRLSCAASGFTFSSYAMSWVRQAPGKGLEWVSAISGSGGSTY YADSVKGRFTISRDNSKNTLYLQMNSLRAEDTAVYYCAKDGGYYDSGAGDYWGQGTL VTVSS (SEQ ID NO:5, ADI-27744). In some embodiments, the antibody heavy chain variable domain is at least 95% identical to SEQ ID NO:5. In some embodiments, the heavy chain variable domain includes amino acid sequences FTFSSYAMS (SEQ ID NO:23) as the first complementarity-determining region (“CDR1”), AISGSGGSTYYADSVKG (SEQ ID NO:24) as the second CDR (“CDR2”), and AKDGGYYDSGAGDY (SEQ ID NO:25) as the third CDR (“CDR3”) of SEQ ID NO:5. In some embodiments, the heavy chain variable domain includes amino acid sequences SYAMS (SEQ ID NO:47) as CDR1, AISGSGGSTYYADSVKG (SEQ ID NO: 24) as CDR2, and DGGYYDSGAGDY (SEQ ID NO:70) as CDR3 of SEQ ID NO:5.

Another aspect of the invention relates to an antibody heavy chain variable domain at least 90% identical to the amino acid sequence EVOLVESGGGLVKPGGSLRLSCAASGFTFSSYSMNWVRQAPGKGLEWVSSISSSSSYIY YADSVKGRFTISRDNAKNSLYLQMNSLRAEDTAVYYCARGAPMGAAAGWFDPWGQG TLVTVSS (SEQ ID NO:7, ADI-27749). In some embodiments, the antibody heavy chain variable domain is at least 95% identical to SEQ ID NO:7. In some embodiments, the heavy chain variable domain includes amino acid sequences FTFSSYSMN (SEQ ID NO:29) as the first complementarity-determining region (“CDR1”), SISSSSSYIYYADSVKG (SEQ ID NO: 30) as the second CDR (“CDR2”), and ARGAPMGAAAGWFDP (SEQ ID NO:31) as the third CDR (“CDR3”) of SEQ ID NO:7. In some embodiments, the heavy chain variable domain includes amino acid sequences SYSMN (SEQ ID NO:48) as CDR1, SISSSSSYIYYADSVKG (SEQ ID NO: 30) as CDR2, and GAPMGAAAGWFDP (SEQ ID NO:71) as CDR3 of SEQ ID NO:7.

Another aspect of the invention relates to an antibody heavy chain variable domain at least 90% identical to the amino acid sequence EVOLVESGGGLVKPGGSLRLSCAASGFTFSSYSMNWVRQAPGKGLEWVSSISSSSSYIY YADSVKGRFTISRDNAKNSLYLQMNSLRAEDTAVYYCARGAPIGAAAGWFDPWGQGT LVTVSS (SEQ ID NO:85, A49MI). In some embodiments, the antibody heavy chain variable domain is at least 95% identical to SEQ ID NO:85. In some embodiments, the heavy chain variable domain includes amino acid sequences FTFSSYSMN (SEQ ID NO:29) as CDR1, SISSSSSYIYYADSVKG (SEQ ID NO: 30) as CDR2, and ARGAPIGAAAGWFDP (SEQ ID NO: 77) as CDR3 of SEQ ID NO:85. In some embodiments, the heavy chain variable domain includes amino acid sequences SYSMN (SEQ ID NO:48) as CDR1, SISSSSSYIYYADSVKG (SEQ ID NO: 30) as CDR2, and GAPIGAAAGWFDP (SEQ ID NO:78) as CDR3 of SEQ ID NO: 85.

Another aspect of the invention relates to an antibody heavy chain variable domain at least 90% identical to the amino acid sequence QVQLVQSGAEVKKPGASVKVSCKASGYTFTSYYMHWVRQAPGQGLEWMGIINPSGGS TSYAQKFQGRVTMTRDTSTSTVYMELSSLRSEDTAVYYCAREGAGFAYGMDYYYMDV WGKGTTVTVSS (SEQ ID NO:9, ADI-29378). In some embodiments, the antibody heavy chain variable domain is at least 95% identical to SEQ ID NO:9. In some embodiments, the heavy chain variable domain includes amino acid sequences YTFTSYYMH (SEQ ID NO:35) as the first complementarity-determining region (“CDR1”), IINPSGGSTSYAQKFQG (SEQ ID NO: 36) as the second CDR (“CDR2”), and AREGAGFAYGMDYYYMDV (SEQ ID NO:37) as the third CDR (“CDR3”) of SEQ ID NO:9. In some embodiments, the heavy chain variable domain includes amino acid sequences SYYMH (SEQ ID NO:45) as CDR1, IINPSGGSTSYAQKFQG (SEQ ID NO: 36) as CDR2, and EGAGFAYGMDYYYMDV (SEQ ID NO: 72) as CDR3 of SEQ ID NO:9.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be included into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, mutations that can be included into the CH1 of a human IgG1 constant region may be at amino acid V125, F126, P127, T135, T139, A140, F170, P171, and/or V173. In certain embodiments, mutations that can be included into the Cκ of a human IgG1 constant region may be at amino acid E123, F116, S176, V163, S174, and/or T164.

In some embodiments, one of the heavy chain variable domains described herein is combined with a light chain variable domain to form an antigen-binding site capable of binding NKG2D. For example, an antibody heavy chain variable domain at least 90% identical to the amino acid sequence of SEQ ID NO: 1 can be paired with an antibody light chain variable domain at least 90% identical to the amino acid sequence EIVMTQSPATLSVSPGERATLSCRASQSVSSNLAWYQQKPGQAPRLLIYGASTRATGIPA RFSGSGSGTEFTLTISSLQSEDFAVYYCQQYDDWPFTFGGGTKVEIK (SEQ ID NO:2, ADI-29379). In some embodiments, the antibody light chain variable domain is at least 95% identical to SEQ ID NO:2. In some embodiments, the light chain variable domain includes amino acid sequences RASQSVSSNLA (SEQ ID NO:14) as the first complementarity-determining region (“CDR”), GASTRAT (SEQ ID NO:15) as the second CDR, and QQYDDWPFT (SEQ ID NO: 16) as the third CDR.

For example, an antibody heavy chain variable domain at least 90% identical to the amino acid sequence of SEQ ID NO:3 can be paired with an antibody light chain variable domain at least 90% identical to the amino acid sequence EIVLTQSPGTLSLSPGERATLSCRASQSVSSNLAWYQQKPGQAPRLLIYGASTRATGIPAR FSGSGSGTEFTLTISSLQSEDFAVYYCQQDDYWPPTFGGGTKVEIK (SEQ ID NO:4, ADI-29463). In some embodiments, the antibody light chain variable domain is at least 95% identical to SEQ ID NO:4. In some embodiments, the light chain variable domain includes amino acid sequences RASQSVSSNLA (SEQ ID NO:20) as the first complementarity-determining region (“CDR”), GASTRAT (SEQ ID NO:21) as the second CDR, and QQDDYWPPT (SEQ ID NO: 22) as the third CDR.

For example, an antibody heavy chain variable domain at least 90% identical to the amino acid sequence of SEQ ID NO:5 can be paired with an antibody light chain variable domain at least 90% identical to the amino acid sequence DIQMTQSPSSVSASVGDRVTITCRASQGIDSWLAWYQQKPGKAPKLLIYAASSLQSGVPS RFSGSGSGTDFTLTISSLQPEDFATYYCQQGVSYPRTFGGGTKVEIK (SEQ ID NO:6, ADI-27744). In some embodiments, the antibody light chain variable domain is at least 95% identical to SEQ ID NO:6. In some embodiments, the light chain variable domain includes amino acid sequences RASQGIDSWLA (SEQ ID NO:26) as the first complementarity-determining region (“CDR”), AASSLOS (SEQ ID NO:27) as the second CDR, and QQGVSYPRT (SEQ ID NO:28) as the third CDR.

For example, an antibody heavy chain variable domain at least 90% identical to the amino acid sequence of SEQ ID NO:7 or 85 can be paired with an antibody light chain variable domain at least 90% identical to the amino acid sequence DIQMTQSPSSVSASVGDRVTITCRASQGISSWLAWYQQKPGKAPKLLIYAASSLQSGVPS RFSGSGSGTDFTLTISSLQPEDFATYYCQQGVSFPRTFGGGTKVEIK (SEQ ID NO:8, ADI-27749). In some embodiments, the antibody light chain variable domain is at least 95% identical to SEQ ID NO:8. In some embodiments, the light chain variable domain includes amino acid sequences RASQGISSWLA (SEQ ID NO:32) as the first complementarity-determining region (“CDR”), AASSLOS (SEQ ID NO:33) as the second CDR, and QQGVSFPRT (SEQ ID NO:34) as the third CDR.

For example, an antibody heavy chain variable domain at least 90% identical to the amino acid sequence of SEQ ID NO:9 can be paired with an antibody light chain variable domain at least 90% identical to the amino acid sequence EIVLTQSPATLSLSPGERATLSCRASQSVSSYLAWYQQKPGQAPRLLIYDASNRATGIPA RFSGSGSGTDFTLTISSLEPEDFAVYYCQQSDNWPFTFGGGTKVEIK (SEQ ID NO:10, ADI-29378). In some embodiments, the antibody light chain variable domain is at least 95% identical to SEQ ID NO:10. In some embodiments, the light chain variable domain includes amino acid sequences RASQSVSSYLA (SEQ ID NO:38) as the first complementarity-determining region (“CDR”), DASNRAT (SEQ ID NO:39) as the second CDR, and QQSDNWPFT (SEQ ID NO:40) as the third CDR.

When a heavy chain variable domain is combined with a light chain variable domain to form an antigen-binding site capable of binding NKG2D, the antigen-binding site can be included into a variety of structures, for example, a typical antibody structure with two identical heavy chains and two identical light chains, forming a pair of antigen-binding sites capable of binding NKG2D; a bi-specific, tri-specific, tetra-specific or other multi-specific antibody; or a smaller structure such as an scFv (in which the heavy chain variable domain is linked to the light chain variable domain).

In some embodiments, any NKG2D antigen-binding site disclosed in the instant invention is included into a protein that also includes a separate antigen-binding site that binds a tumor-associated antigen, which may permit the protein to simultaneously interact with an NK cell and a tumor cell. The tumor-associated antigen, for example, can be CD33, HER2, EpCAM, CD2, CD3, CD8, CD10, CD19, CD20, CD21, CD22, CD23, CD24, CD25, CD30, CD37, CD38, CD40, CD45RO, CD48, CD52, CD55, CD59, CD70, CD74, CD80, CD86, CD138, CD147, HLA-DR, CSAP, CA-125, TAG-72, EFGR/ERBB1, IGF1R, HER2, HER3, HER4, IGF-1R, c-Met, PDGFR, MUC1, MUC2, MUC3, MUC4, TNFR1, TNFR2, NGFR, TRAILR1, TRAILR2, Fas (CD95), DR3, DR4, DR5, DR6, VEGF, PIGF, tenascin, ED-B fibronectin, PSA, and IL-6, MAGE-A3, B7.1, B7.2, CTLA4 or PD1.

In some embodiments, any NKG2D antigen-binding site disclosed in the instant invention is included into a protein that also contain a tumor-associated antigen site and CD16 binding site. The CD16 binding site can be an additional antigen-binding site or an antibody constant region or a portion thereof, such as an IgG1 constant region (which may optionally include one or more mutations affecting, for example, effector activity or CD16 binding affinity).

Another aspect of the invention provides a method of enhancing tumor cell death and treating cancer in a patient. The method comprises administering to a patient in need thereof a therapeutically effective amount of a protein described herein to treat the cancer.

### BRIEF DESCRIPTION OF THE DRAWINGS

FIG. 1 is a representation of a multispecific binding protein that contains an NKG2D-binding domain (right arm), a tumor-associated antigen-binding domain (left arm) and an Fc domain or a portion thereof that binds to CD16.

FIG. 2 is a representation of a multispecific binding protein that includes a NKG2D-binding domain or a tumor-associated antigen-binding domain, either one of which can be in an scFv format, and an Fc domain or a portion thereof that binds to CD16.

FIGS. 3A-3E are profiles of NKG2D-binding affinity of the NKG2D-binding domains measured by surface plasmon resonance. FIG. 3A is the NKG2D-binding affinity of the NKG2D-binding domain ADI-27744 measured by surface plasmon resonance; FIG. 3B is the NKG2D-binding affinity of the NKG2D-binding domain ADI-29379 measured by surface plasmon resonance; FIG. 3C is the NKG2D-binding affinity of the NKG2D-binding domain ADI-27749 measured by surface plasmon resonance; FIG. 3D is the NKG2D-binding affinity of the NKG2D-binding domain ADI-29463 measured by surface plasmon resonance; and FIG. 3E is the NKG2D-binding affinity of the NKG2D-binding domain ADI-29378 measured by surface plasmon resonance.

FIGS. 4A-H are profiles of competitive NKG2D binding by NKG2D-binding domain ADI-27744 (A44) and ULBP6 or other NKG2D antibodies measured by surface plasmon resonance. FIG. 4A shows the profile of NKG2D monoclonal antibody comprising ADI-27744 injected over immobilized NKG2D, followed by injection of ULBP6. FIG. 4B shows the profile of ULBP6 injected over immobilized NKG2D, followed by injection of NKG2D monoclonal antibody comprising ADI-27744. FIG. 4C shows the profile of MS monoclonal antibody injected over immobilized NKG2D, followed by injection of ULBP6. FIG. 4D shows the profile of MS injected over the immobilized NKG2D, followed by injection of NKG2D monoclonal antibody comprising ADI-27744. FIG. 4E shows the profile of 1D11 injected over the immobilized NKG2D, followed by injection of NKG2D monoclonal antibody comprising ADI-27744. FIG. 4F shows the profile of MAB139 injected over the immobilized NKG2D, followed by injection of NKG2D monoclonal antibody comprising ADI-27744. FIG. 4G shows the profile of NKG2D monoclonal antibody comprising ADI-27744 was injected over the immobilized NKG2D, followed by injection of NKG2D monoclonal antibody comprising ADI-27749 (A49); and FIG. 4H shows the profile of NKG2D monoclonal antibody comprising ADI-27744 was injected over the immobilized NKG2D, followed by injection of NKG2D monoclonal antibody comprising F47.

FIG. 5 are line graphs showing the binding profile of CD33-targeting TriNKETs to NKG2D expressed on EL4 cells.

FIG. 6 are line graphs showing the binding profile of HER2-targeting TriNKETs to NKG2D expressed on EL4 cells.

FIG. 7 are bar graphs showing the binding profile of CD33-targeting TriNKETs to CD33 expressed on Mv4-11 cells.

FIG. 8 are bar graphs showing the binding profile of HER2-targeting TriNKETs to HER2 expressed on 786-O cells.

FIG. 9 are bar graphs showing the binding profile of a HER2-targeting TriNKETs to HER2 expressed on NCI-H661 cells.

FIG. 10 are bar graphs showing that HER2-targeting TriNKETs mediate activation of human NK cells co-cultured with HER2-expressing NCI-H661 cells.

FIG. 11 are bar graphs showing that HER2-targeting TriNKETs mediate activation of human NK cells co-cultured with HER2 expressing SkBr-3 cells.

FIG. 12 are bar graphs showing that CD33-targeting TriNKETs mediate activation of human NK cells co-cultured with CD33-expressing human AML Mv4-11 cells.

FIG. 13 are line graphs showing that CD33-targeting TriNKETs enable cytotoxicity of rested NK cells against CD33-expressing Molm-13 cancer cells.

FIG. 14 are line graphs showing that CD33-targeting TriNKETs enable cytotoxicity of activated NK cells against CD33-expressing Molm-13 cancer cells.

FIG. 15 are bar graphs showing that HER2-targeting TriNKETs enable cytotoxicity of rested NK cells against HER2-expressing 786-O cancer cells.

FIG. 16 are bar graphs showing that HER2-targeting TriNKETs enable cytotoxicity of activated NK cells against HER2-expressing 786-O cancer cells.

FIG. 17 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form is an heterodimeric construct containing ½ of rat antibody and ½ of mouse antibody.

FIG. 18 is a representation of a TriNKET in the KiH Common Light Chain (LC) form, which involves the knobs-into-holes (KIHs) technology. KiH is a heterodimer containing 2 Fabs binding to target 1 and 2, and an Fc stabilized by heterodimerization mutations. TriNKET in the KiH format may be an heterodimeric construct with 2 fabs binding to target 1 and target 2, containing 2 different heavy chains and a common light chain that pairs with both HC.

FIG. 19 is a representation of a TriNKET in the dual-variable domain immunoglobulin (DVD-Ig™) form, which combines the target binding domains of two monoclonal antibodies via flexible naturally occurring linkers, and yields a tetravalent IgG-like molecule. DVD-Ig™ is an homodimeric construct where variable domain targeting antigen 2 is fused to the N terminus of variable domain of Fab targeting antigen 1 Construct contains normal Fc.

FIG. 20 is a representation of a TriNKET in the Orthogonal Fab interface (Ortho-Fab) form, which is an heterodimeric construct that contains 2 Fabs binding to target1 and target2 fused to Fc. LC-HC pairing is ensured by orthogonal interface. Heterodimerization is ensured by mutations in the Fc.

FIG. 21 is a representation of a TrinKET in the 2-in-1 Ig format.

FIG. 22 is a representation of a TriNKET in the ES form, which is an heterodimeric construct containing 2 different Fabs binding to target 1 and target 2 fused to the Fc. Heterodimerization is ensured by electrostatic steering mutations in the Fc.

FIG. 23 is a representation of a TriNKET in the Fab Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fabs binding to target 1 and 2, and an Fc stabilized by heterodimerization mutations.

FIG. 24 is a representation of a TriNKET in the SEED Body form, which is an heterodimer containing 2 Fabs binding to target 1 and 2, and an Fc stabilized by heterodimerization mutations.

FIG. 25 is a representation of a TriNKET in the LuZ-Y form, in which leucine zipper is used to induce heterodimerization of two different HCs. LuZ-Y form is a heterodimer containing 2 different scFabs binding to target 1 and 2, fused to Fc. Heterodimerization is ensured through leucine zipper motifs fused to C-terminus of Fc.

FIG. 26 is a representation of a TriNKET in the Cov-X-Body form.

FIGS. 27A-27B are representations of TriNKETs in the κλ-Body forms, which are an heterodimeric constructs with 2 different Fabs fused to Fc stabilized by heterodimerization mutations: Fab1 targeting antigen 1 contains kappa LC, while second Fab targeting antigen 2 contains lambda LC. FIG. 27A is an exemplary representation of one form of a κλ-Body; FIG. 27B is an exemplary representation of another κλ-Body.

FIG. 28 is an Oasc-Fab heterodimeric construct that includes Fab binding to target 1 and scFab binding to target 2 fused to Fc. Heterodimerization is ensured by mutations in the Fc.

FIG. 29 is a DuetMab, which is an heterodimeric construct containing 2 different Fabs binding to antigen 1 and 2 and Fc stabilized by heterodimerization mutations. Fab 1 and 2 contain differential S-S bridges that ensure correct LC and HC pairing.

FIG. 30 is a CrossmAb, which is an heterodimeric construct with 2 different Fabs binding to Target 1 and 2 fused to Fc stabilized by heterodimerization. CL and CH1 domains and VH and VL domains are switched, e.g., CH1 is fused in-line with VL, while CL is fused in-line with VH.

FIG. 31 is a Fit-Ig, which is an homodimeric constructs where Fab binding to antigen 2 is fused to the N terminus of HC of Fab that binds to antigen 1. The construct contains wild-type Fc.

FIG. 32 is a series of line graphs showing the binding of TriNKET A* and TriNKET A to human NKG2D as tested by SPR. The upper panels represent kinetic fit, and the lower panels represent steady state affinity fit.

FIG. 33 is a line graph showing the potency of TriNKET A and TriNKET A* in mediating cytotoxicity of NK cells against target cells.

FIG. 34 is a line graph showing the potency of TriNKET A and TriNKET A* in mediating cytotoxicity of NK cells against target cells.

### DETAILED DESCRIPTION

The invention provides antibody heavy chain variable domains that can be paired with antibody light chain variable domains to form an antigen-binding site targeting the NKG2D receptor on natural killer cells, proteins that include the NKG2D antigen-binding sites, pharmaceutical compositions comprising such proteins, and therapeutic methods using such proteins and pharmaceutical compositions for the treatment of cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### NKG2D Antigen-Binding Site

The invention provides antigen-binding sites that bind NKG2D, and antigen heavy chain variable domains that can be used to create such antigen-binding sites.

Antibody heavy chain variable domains and the light chain variable domains which they pair to form antigen-binding sites capable of binding and agonizing the NKG2D receptor have now been identified and are provided in Table 1, below. Unless otherwise indicated, the CDR sequences provided in Table 1 are determined under Kabat.

TABLE 1

| | Heavy chain variable region | Light chain variable region |
|---|---|---|
| Clones | amino acid sequence | amino acid sequence |
| ADI-29379 | QVQLVQSGAEVKKPGASVKVSCKA | EIVMTQSPATLSVSPGERATLSC |
| (E79) | SGYTFTSYYMHWVRQAPGQGLEW | RASQSVSSNLAWYQQKPGQAP |
| | MGIINPSGGSTSYAQKFQGRVTMTR | RLLIYGASTRATGIPARFSGSGS |
| | DTSTSTVYMELSSLRSEDTAVYYCA | GTEFTLTISSLQSEDFAVYYCQQ |
| | RGAPNYGDTTHDYYYMDVWGKGT | YDDWPFTFGGGTKVEIK |
| | TVTVSS | (SEQ ID NO: 2) |
| | (SEQ ID NO: 1) | CDR1 (SEQ ID NO: 14) - |
| | CDR1 non-Kabat (SEQ ID NO: 11) - | RASQSVSSNLA |
| | YTFTSYYMH or CDR1 (SEQ ID | CDR2 (SEQ ID NO: 15) - |
| | NO: 45) - SYYMH | GASTRAT |
| | CDR2 (SEQ ID NO: 12) - | CDR3 (SEQ ID NO: 16) - |
| | IINPSGGSTSYAQKFQG | QQYDDWPFT |
| | CDR3 non-Kabat (SEQ ID NO: 13) - | |
| | ARGAPNYGDTTHDYYYMDV or | |
| | CDR3 (SEQ ID NO: 68) - | |
| | GAPNYGDTTHDYYYMDV | |
| ADI-29463 | QVQLVQSGAEVKKPGASVKVSCKA | EIVLTQSPGTLSLSPGERATLSCR |
| (F63) | SGYTFTGYYMHWVRQAPGQGLEW | ASQSVSSNLAWYQQKPGQAPRL |
| | MGWINPNSGGTNYAQKFQGRVTMT | LIYGASTRATGIPARFSGSGSGT |
| | RDTSISTAYMELSRLRSDDTAVYYC | EFTLTISSLQSEDFAVYYCQQDD |
| | ARDTGEYYDTDDHGMDVWGQGTT | YWPPTFGGGTKVEIK |
| | VTVSS | (SEQ ID NO: 4) |
| | (SEQ ID NO: 3) | CDR1 (SEQ ID NO: 20) - |
| | CDR1 non-Kabat (SEQ ID NO: 17) - | RASQSVSSNLA |
| | YTFTGYYMH or CDR1 (SEQ ID | CDR2 (SEQ ID NO: 21) - |
| | NO: 46) - GYYMH | GASTRAT |
| | CDR2 (SEQ ID NO: 18) - | CDR3 (SEQ ID NO: 22) - |
| | WINPNSGGTNYAQKFQG | QQDDYWPPT |
| | CDR3 non-Kabat (SEQ ID NO: 19) - | |
| | ARDTGEYYDTDDHGMDV or CDR3 | |
| | (SEQ ID NO: 69) - | |
| | DTGEYYDTDDHGMDV | |
| ADI-27744 | EVQLLESGGGLVQPGGSLRLSCAAS | DIQMTQSPSSVSASVGDRVTITC |
| (A44) | GFTFSSYAMSWVRQAPGKGLEWVS | RASQGIDSWLAWYQQKPGKAP |
| | AISGSGGSTYYADSVKGRFTISRDNS | KLLIYAASSLQSGVPSRFSGSGS |
| | KNTLYLQMNSLRAEDTAVYYCAKD | GTDFTLTISSLQPEDFATYYCQQ |
| | GGYYDSGAGDYWGQGTLVTVSS | GVSYPRTFGGGTKVEIK |
| | (SEQ ID NO: 5) | (SEQ ID NO: 6) |
| | CDR1 non-Kabat (SEQ ID NO: 23) - | CDR1 (SEQ ID NO: 26) - |
| | FTFSSYAMS or CDR1 (SEQ ID | RASQGIDSWLA |
| | NO: 47) - SYAMS | |
| | CDR2 (SEQ ID NO: 24) - | CDR2 (SEQ ID NO: 27) - |
| | AISGSGGSTYYADSVKG | AASSLQS |
| | CDR3 non-Kabat (SEQ ID NO: 25) - | CDR3 (SEQ ID NO: 28) - |
| | AKDGGYYDSGAGDY or CDR3 (SEQ | QQGVSYPRT |
| | ID NO: 70) - DGGYYDSGAGDY | |
| ADI-27749 | EVQLVESGGGLVKPGGSLRLSCAAS | DIQMTQSPSSVSASVGDRVTITC |
| (A49) | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PMGAAAGWFDPWGQGTLVTVSS | GVSFPRTFGGGTKVEIK |
| | (SEQ ID NO: 7) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 31) - | QQGVSFPRT |
| | ARGAPMGAAAGWFDP or CDR3 | |
| | (SEQ ID NO: 71) - | |
| | GAPMGAAAGWFDP | |
| ADI-29378 | QVQLVQSGAEVKKPGASVKVSCKA | EIVLTQSPATLSLSPGERATLSCR |
| (E78) | SGYTFTSYYMHWVRQAPGQGLEW | ASQSVSSYLAWYQQKPGQAPRL |
| | MGIINPSGGSTSYAQKFQGRVTMTR | LIYDASNRATGIPARFSGSGSGT |
| | DTSTSTVYMELSSLRSEDTAVYYCA | DFTLTISSLEPEDFAVYYCQQSD |
| | REGAGFAYGMDYYYMDVWGKGTT | NWPFTFGGGTKVEIK |
| | VTVSS | (SEQ ID NO: 10) |
| | (SEQ ID NO: 9) | CDR1 (SEQ ID NO: 38) - |
| | CDR1 non-Kabat (SEQ ID NO: 35) - | RASQSVSSYLA |
| | YTFTSYYMH or CDR1 (SEQ ID | CDR2 (SEQ ID NO: 39) - |
| | NO: 45) - SYYMH | DASNRAT |
| | CDR2 (SEQ ID NO: 36) - | CDR3 (SEQ ID NO: 40) - |
| | IINPSGGSTSYAQKFQG | QQSDNWPFT |
| | CDR3 non-Kabat (SEQ ID NO: 37) - | |
| | AREGAGFAYGMDYYYMDV or | |
| | CDR3 (SEQ ID NO: 72) - | |
| | EGAGFAYGMDYYYMDV | |
| A49MQ | EVQLVESGGGLVKPGGSLRLSCAAS | DIQMTQSPSSVSASVGDRVTITC |
| | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PQGAAAGWFDPWGQGTLVTVSS | GVSFPRTFGGGTKVEIK |
| | (SEQ ID NO: 83) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 73) - | QQGVSFPRT |
| | ARGAPQGAAAGWFDP or CDR3 | |
| | (SEQ ID NO: 74) - | |
| | GAPQGAAAGWFDP | |
| A49ML | EVQLVESGGGLVKPGGSLRLSCAAS | DIQMTQSPSSVSASVGDRVTITC |
| | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PLGAAAGWFDPWGQGTLVTVSS | GVSFPRTFGGGTKVEIK |
| | (SEQ ID NO: 84) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 75) - | QQGVSFPRT |
| | ARGAPLGAAAGWFDP or CDR3 | |
| | (SEQ ID NO: 76) - | |
| | GAPLGAAAGWFDP | |
| A49MI | EVQLVESGGGLVKPGGSLRLSCAAS | DIQMTQSPSSVSASVGDRVTITC |
| | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PIGAAAGWFDPWGQGTLVTVSS | GVSFPRTFGGGTKVEIK |
| | (SEQ ID NO: 85) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 77) - | QQGVSFPRT |
| | ARGAPIGAAAGWFDP or CDR3 (SEQ | |
| | ID NO: 78) - GAPIGAAAGWFDP | |
| A49MF | EVQLVESGGGLVKPGGSLRLSCAAS | DIQMTQSPSSVSASVGDRVTITC |
| | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PFGAAAGWFDPWGQGTLVTVSS | GVSFPRTFGGGTKVEIK |
| | (SEQ ID NO: 86) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 79) - | QQGVSFPRT |
| | ARGAPFGAAAGWFDP or CDR3 | |
| | (SEQ ID NO: 80) - | |
| | GAPFGAAAGWFDP | |
| A49MV | EVQLVESGGGLVKPGGSLRLSCAAS | DIQMTQSPSSVSASVGDRVTITC |
| | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PVGAAAGWFDPWGQGTLVTVSS | GVSFPRTFGGGTKVEIK |
| | (SEQ ID NO: 41) | (SEQ ID NO: 8) |
| | CDR1 non-Kabat (SEQ ID NO: 29) - | CDR1 (SEQ ID NO: 32) - |
| | FTFSSYSMN or CDR1 (SEQ ID | RASQGISSWLA |
| | NO: 48) - SYSMN | CDR2 (SEQ ID NO: 33) - |
| | CDR2 (SEQ ID NO: 30) - | AASSLQS |
| | SISSSSSYIYYADSVKG | CDR3 (SEQ ID NO: 34) - |
| | CDR3 non-Kabat (SEQ ID NO: 81) - | QQGVSFPRT |
| | ARGAPVGAAAGWFDP or CDR3 | |
| | (SEQ ID NO: 82) - | |
| | GAPVGAAAGWFDP | |
| A49- | EVQLVESGGGLVKPGGSLRLSCAAS | DIQMTQSPSSVSASVGDRVTITC |
| consensus | GFTFSSYSMNWVRQAPGKGLEWVS | RASQGISSWLAWYQQKPGKAP |
| | SISSSSSYIYYADSVKGRFTISRDNAK | KLLIYAASSLQSGVPSRFSGSGS |
| | NSLYLQMNSLRAEDTAVYYCARGA | GTDFTLTISSLQPEDFATYYCQQ |
| | PXGAAAGWFDPWGQGTLVTVSS, | GVSFPRTFGGGTKVEIK |
| | wherein X is M, L, I, V, Q, or F | (SEQ ID NO: 8) |
| | (SEQ ID NO: 42) | CDR1 (SEQ ID NO: 32) - |
| | CDR1 non-Kabat (SEQ ID NO: 29) - | RASQGISSWLA |
| | FTFSSYSMN or CDR1 (SEQ ID | CDR2 (SEQ ID NO: 33) - |
| | NO: 48) - SYSMN | AASSLQS |
| | CDR2 (SEQ ID NO: 30) - | CDR3 (SEQ ID NO: 34) - |
| | SISSSSSYIYYADSVKG | QQGVSFPRT |
| | CDR3 non-Kabat (SEQ ID NO: 43) - | |
| | ARGAPXGAAAGWFDP or CDR3 | |
| | (SEQ ID NO: 44) - | |
| | GAPXGAAAGWFDP, wherein X is M, | |
| | L, I, V, Q, or F | |

One advantage of one or more of the antibody heavy chain variable domain amino acid sequences described above is that they can bind to NKG2D from humans and cynomolgus monkeys to agonize the receptor, and compete with natural ligands for binding to the receptor. Additional antigen-binding sites that bind NKG2D and share one or more of these properties are also particularly useful and can be identified by binding competition assays known in the art. For example, the additional antigen-binding sites can be identified by competition with ADI-29379, ADI-29463, ADI-27744, ADI-27749, or ADI-29378 for binding to both human and optionally cynomolgus monkey NKG2D.

Another advantage of the NKG2D-binding sites which comprise the antibody heavy chain variable domains and light chain variable domains sequences described above is that they can bind to NKG2D with high affinity. In some embodiments, NKG2D-binding sites bind to NKG2D with a K_{D} of 0.1 to 1000 nM. In some embodiments, NKG2D-binding sites bind to NKG2D with a K_{D} of 1 to 500 nM. In some embodiments, NKG2D-binding sites bind to NKG2D with a K_{D} of 5 to 100 nM. In some embodiments, NKG2D-binding sites bind to NKG2D with a K_{D} of 10 to 62 nM.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:1, and an antibody light chain variable domain that includes an amino acid sequence at least 90% (e.g., 91%, 92%, 93%, 94%, 95%, 96%, 97%, 98%, 99%, or 100%) identical to SEQ ID NO:2. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO: 1, includes a CDR1 sequence represented by the amino acid sequence of SEQ ID NO: 11 or SEQ ID NO:91, a CDR2 sequence represented by the amino acid sequence of SEQ ID NO: 12, and a CDR3 sequence represented by the amino acid sequence of SEQ ID NO:13 or 68. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:2, includes a CDR1 sequence represented by the amino acid sequence of SEQ ID NO:14, a CDR2 sequence represented by the amino acid sequence of SEQ ID NO: 15, and a CDR3 sequence represented by the amino acid sequence of SEQ ID NO:16.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:3, and an antibody light chain variable domain that includes an amino acid sequence at least 90% (e.g., 91%, 92%, 93%, 94%, 95%, 96%, 97%, 98%, 99%, or 100%) identical to SEQ ID NO:4. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:3, includes a CDR1 sequence represented by the amino acid sequence of SEQ ID NO: 17 or SEQ ID NO:92, a CDR2 sequence represented by the amino acid sequence of SEQ ID NO: 18, and a CDR3 sequence represented by the amino acid sequence of SEQ ID NO:19 or SEQ ID NO: 69. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:4, includes a CDR1 sequence represented by the amino acid sequence of SEQ ID NO:20, a CDR2 sequence represented by the amino acid sequence of SEQ ID NO:21, and a CDR3 sequence represented by the amino acid sequence of SEQ ID NO:22.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., 91%, 92%, 93%, 94%, 95%, 96%, 97%, 98%, 99%, or 100%) identical to SEQ ID NO:6. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:5, includes a CDR1 sequence represented by the amino acid sequence of SEQ ID NO: 23 or SEQ ID NO:93, a CDR2 sequence represented by the amino acid sequence of SEQ ID NO: 24, and a CDR3 sequence represented by the amino acid sequence of SEQ ID NO:25 or SEQ ID NO: 70. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:6, includes a CDR1 sequence represented by the amino acid sequence of SEQ ID NO:26, a CDR2 sequence represented by the amino acid sequence of SEQ ID NO:27, and a CDR3 sequence represented by the amino acid sequence of SEQ ID NO:28.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:7, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:7, includes a CDR1 sequence represented by the amino acid sequence of SEQ ID NO: 29 or SEQ ID NO:94, a CDR2 sequence represented by the amino acid sequence of SEQ ID NO: 30, and a CDR3 sequence represented by the amino acid sequence of SEQ ID NO:31 or SEQ ID NO: 71. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

The amino acid residue M at position 102 of SEQ ID NO:7, which is in CDR3 of the heavy chain variable domain, can be mutated. In certain embodiments, M102 is substituted by a non-charged residue. In certain embodiments, M102 is substituted by a hydrophobic residue (Gly, Ala, Val, Leu, Ile, Pro, Phe, or Trp). In certain embodiments, M102 is substituted by a polar residue (Ser, Thr, Cys, Asn, Gln, or Tyr). In certain embodiments, M102 is substituted by Leu, Ile, Val, Gln, or Phc.

Accordingly, in certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:83, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:83, includes a CDR1 sequence represented by the amino acid sequence of SEQ ID NO:29 or SEQ ID NO:94, a CDR2 sequence represented by the amino acid sequence of SEQ ID NO:30, and a CDR3 sequence represented by the amino acid sequence of SEQ ID NO:73 or SEQ ID NO:74. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:84, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:84, includes a CDR1 sequence represented by the amino acid sequence of SEQ ID NO: 29 or SEQ ID NO:94, a CDR2 sequence represented by the amino acid sequence of SEQ ID NO: 30, and a CDR3 sequence represented by the amino acid sequence of SEQ ID NO:75 or SEQ ID NO: 76. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:85, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:85, includes a CDR1 sequence represented by the amino acid sequence of SEQ ID NO: 29 or SEQ ID NO:94, a CDR2 sequence represented by the amino acid sequence of SEQ ID NO: 30, and a CDR3 sequence represented by the amino acid sequence of SEQ ID NO:77 or SEQ ID NO: 78. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:86, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:86, includes a CDR1 sequence represented by the amino acid sequence of SEQ ID NO: 29 or SEQ ID NO:94, a CDR2 sequence represented by the amino acid sequence of SEQ ID NO: 30, and a CDR3 sequence represented by the amino acid sequence of SEQ ID NO:79 or 80. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:41, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:41, includes a CDR1 sequence represented by the amino acid sequence of SEQ ID NO: 29 or SEQ ID NO:94, a CDR2 sequence represented by the amino acid sequence of SEQ ID NO: 30, and a CDR3 sequence represented by the amino acid sequence of SEQ ID NO:81 or SEQ ID NO: 82. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR I sequence represented by the amino acid sequence of SEQ ID NO:32, a CDR2 sequence represented by the amino acid sequence of SEQ ID NO:33, and a CDR3 sequence represented by the amino acid sequence of SEQ ID NO:34.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:9, and an antibody light chain variable domain that includes an amino acid sequence at least 90% (e.g., 91%, 92%, 93%, 94%, 95%, 96%, 97%, 98%, 99%, or 100%) identical to SEQ ID NO:10. In certain embodiments, an antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:9, includes a CDR1 sequence represented by the amino acid sequence of SEQ ID NO: 35 or SEQ ID NO:91, a CDR2 sequence represented by the amino acid sequence of SEQ ID NO: 36, and a CDR3 sequence represented by the amino acid sequence of SEQ ID NO:37 or SEQ ID NO: 72. In certain embodiments, an antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:10, includes a CDR1 sequence represented by the amino acid sequence of SEQ ID NO:38, a CDR2 sequence represented by the amino acid sequence of SEQ ID NO:39, and a CDR3 sequence represented by the amino acid sequence of SEQ ID NO:40.

In certain embodiments, the present invention provides an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., 91%, 92%, 93%, 94%, 95%, 96%, 97%, 98%, 99%, or 100%) identical to SEQ ID NO:6, that does not block the binding of anti-NKG2D antibodies MS, 1D11, and MAB139 to NKG2D.

In embodiments, an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:7, 83, 84, 85, 86, or 87, and an antibody light chain variable domain that includes an amino acid sequence at least 90% (e.g., 91%, 92%, 93%, 94%, 95%, 96%, 97%, 98%, 99%, or 100%) identical to SEQ ID NO:8, does not block binding of an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., 91%, 92%, 93%, 94%, 95%, 96%, 97%, 98%, 99%, or 100%) identical to SEQ ID NO:6 to NKG2D.

In certain embodiments, an antigen-binding site that includes an antibody heavy chain variable domain that includes an amino acid sequence at least 90% (e.g., 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., 91%, 92%, 93%, 94%, 95%, 96%, 97%, 98%, 99%, or 100%) identical to SEQ ID NO:6, binds to a unique epitope on NKG2D, different from MS, 1D11, MAB139, ADI-27749 and F47 binding epitope(s).

### Antibodies and Multi-Specific Binding Proteins

In some embodiments of this invention, the NKG2D antigen-binding sites formed by pairing an antibody heavy chain variable domain with a light chain variable domain described herein can be included into larger proteins such as intact antibodies, multi-specific binding proteins or multi-specific binding antibodies. For example, an NKG2D-binding site can be combined with a second component, e.g., a second antigen-binding site. In some embodiments, the second antigen-binding site binds to one or more tumor-associated antigens, such as CD33, HER2, EpCAM, CD2, CD3, CD8, CD10, CD19, CD20, CD21, CD22, CD23, CD24, CD25, CD30, CD33, CD37, CD38, CD40, CD45RO, CD48, CD52, CD55, CD59, CD70, CD74, CD80, CD86, CD138, CD147, HLA-DR, CSAp, CA-125, TAG-72, EFGR/ERBB1, IGF1R, HER3, HER4, IGF-1R, c-Mct, PDGFR, MUC1, MUC2, MUC3, MUC4, TNFR1, TNFR2, NGFR, TRAILR1, TRAILR2, Fas (CD95), DR3, DR4, DR5, DR6, VEGF, PIGF, tenascin, ED-B fibronectin, PSA, and IL-6, MAGE-A3, B7.1, B7.2, CTLA4 or PD1. Binding of a multi-specific protein to NKG2D and to a tumor-associated antigen on a cancer cell brings the cancer cell into proximity to the natural killer cell, which facilitates destruction of the cancer cell by the natural killer cell either directly or indirectly.

In some embodiments, in addition to an NKG2D-binding site and a tumor-associated antigen-binding site, a multi-specific binding protein can further include a domain that binds to CD16, an Fc receptor on the surface of leukocytes including natural killer cells, macrophages, neutrophils, eosinophils, mast cells, and follicular dendritic cells. In some embodiments, the CD16 binding domain can include an antibody Fc region or a portion thereof. In some embodiments, the domain that binds to CD16 contains hinge, CH2 and CH3 domains of an antibody Fc region without or without CH1 domain. In some embodiments, the antibody Fc region is derived from the Fc regions in the human and/or other mammalian immunoglobulins. It is known that within the Fc region, CD16 binding is mediated by the hinge region and the CH2 domain. For example, within human IgG1, the interaction with CD16 is mediated through amino acid residues Asp 265-Glu 269, Asn 297-Thr 299, Ala 327-Ile 332, Leu 234-Ser 239, and carbohydrate residue N-acetyl-D-glucosamine in the CH2 domain (see Sondermann et al, Nature, 406(6793):267-273). Based on the known domains and amino acid residues, in some embodiments, mutations can be selected within the CD16 binding domain to enhance or reduce its binding affinity to CD16. Selection methods are well known methods in the art, such as phage-displayed libraries or yeast surface-displayed cDNA libraries. Appropriate selection methods can also be designed based on the known three-dimensional structure of the interaction by a skilled person in the art.

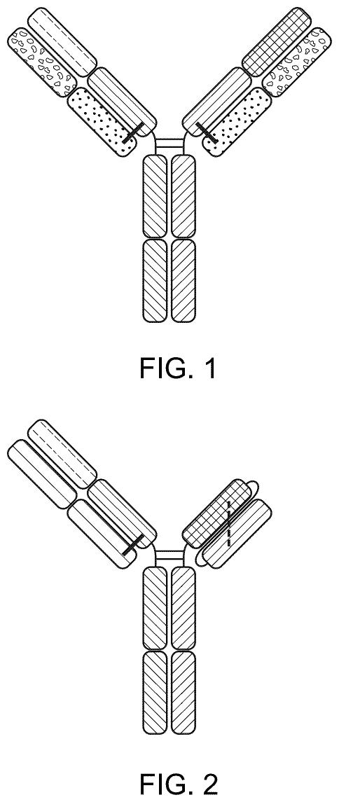

The multi-specific binding proteins described herein can take various formats. For example, one format is a heterodimeric, multi-specific antibody that includes a first immunoglobulin heavy chain, a first immunoglobulin light chain, a second immunoglobulin heavy chain and a second immunoglobulin light chain. The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. The second immunoglobulin light chain, together with the second immunoglobulin heavy chain, forms an antigen-binding site that binds a tumor antigen. The first Fc domain and second Fc domain together are able to bind to CD16 (FIG. 1).

Another exemplary format involves a heterodimeric, multi-specific antibody that includes a first immunoglobulin heavy chain, a second immunoglobulin heavy chain and an immunoglobulin light chain. The first immunoglobulin heavy chain includes a first Fc (hinge-CH2-CH3) domain fused via either a linker or an antibody hinge to a single-chain variable fragment (scFv) composed of a heavy variable domain and light chain variable domain which pair and bind NKG2D. The second immunoglobulin heavy chain includes a second Fc (hinge-CH2-CH3) domain, a second heavy chain variable domain and optionally a CH1 heavy chain domain. The immunoglobulin light chain includes a light chain variable domain and a constant light chain domain. The second immunoglobulin heavy chain pairs with the immunoglobulin light chain and binds to a tumor-associated antigen. The first Fc domain and the second Fc domain together are able to bind to CD16 (FIG. 2). Additional formats of the multi-specific binding proteins can be devised by combining various formats of NKG2D-binding-fragments described herein.

One or more additional binding motifs may be fused to the C-terminus of the constant region CH3 domain, optionally via a linker sequence. In certain embodiments, the antigen-binding site could be a single-chain or disulfide-stabilized variable region (scFv) or could form a tetravalent or trivalent molecule.

In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies. Triomab form is an heterodimeric construct containing ½ of rat antibody and ½ of mouse antibody.

In some embodiments, the multi-specific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (i.e., T366W_{CH3A} in EU numbering). To accommodate the “knob,” a complementary “hole” surface was created on the other CH3 domain (CH3B) by replacing the closest neighboring residues to the knob with smaller ones (i.e., T366S/L368A/Y407V_{CH3B}). The “hole” mutation was optimized by structured-guided phage library screening (Atwell S, Ridgway JB, Wells JA, Carter P. Stable heterodimers from remodeling the domain interface of a homodimer using a phage display library. J Mol Biol (1997) 270(1):26-35). X-ray crystal structures of KiH Fc variants (Elliott JM, Ultsch M, Lee J, Tong R, Takeda K, Spiess C, et al., Antiparallel conformation of knob and hole aglycosylated half-antibody homodimers is mediated by a CH2-CH3 hydrophobic interaction. J Mol Biol (2014) 426(9):1947-57; Mimoto F, Kadono S, Katada H, Igawa T, Kamikawa T, Hattori K. Crystal structure of a novel asymmetrically engineered Fc variant with improved affinity for FcgammaRs. Mol Immunol (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule. DVD-Ig™ is an homodimeric construct where variable domain targeting antigen 2 is fused to the N terminus of variable domain of Fab targeting antigen 1 Construct contains normal Fc.

In some embodiments, the multi-specific binding protein is in the Orthogonal Fab interface (Ortho-Fab) form, which is an heterodimeric construct that contains 2 Fabs binding to target 1 and target 2 fused to Fc. LC-HC pairing is ensured by orthogonal interface. Heterodimerization is ensured by mutations in the Fc. In ortho-Fab IgG approach (Lewis SM, Wu X, Pustilnik A, Sereno A, Huang F, Rick HL, et al. Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab, without any changes being made to the other Fab.

In some embodiments, the multi-specific binding protein is in the 2-in-1 Ig format. In some embodiments, the multi-specific binding protein is in the ES form, which is an heterodimeric construct containing 2 different Fabs binding to target 1 and target 2 fused to the Fc. Heterodimerization is ensured by electrostatic steering mutations in the Fc.

In some embodiments, the multi-specific binding protein is in the κλ-Body form, which are an heterodimeric constructs with 2 different Fabs fused to Fc stabilized by heterodimerization mutations: Fab1 targeting antigen 1 contains kappa LC, while second Fab targeting antigen 2 contains lambda LC.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies). Fab Arm Exchange form (cFae) is a heterodimer containing 2 Fabs binding to target 1 and 2, and an Fc stabilized by heterodimerization mutations.

In some embodiments, the multi-specific binding protein is in the SEED Body form which is an heterodimer containing 2 Fabs binding to target 1 and 2, and an Fc stabilized by heterodimerization mutations. The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which leucine zipper is used to induce heterodimerization of two different HCs. (Wranik, BJ. et al., J. Biol. Chem. (2012), 287:43331-9). LuZ-Y form is a heterodimer containing 2 different scFabs binding to target 1 and 2, fused to Fc. Heterodimerization is ensured through leucine zipper motifs fused to C-terminus of Fc.

In some embodiments, the multi-specific binding protein is in the Cov-X-Body form (In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi VR et al., PNAS (2010), 107(52); 22611-22616).

In some embodiments, the multi-specific binding protein is in an Oasc-Fab heterodimeric format that includes Fab binding to target 1 and scFab binding to target 2 fused to Fc. Heterodimerization is ensured by mutations in the Fc.

In some embodiments, the multi-specific binding protein is in an DuetMab format containing 2 different Fabs binding to antigen 1 and 2 and Fc stabilized by heterodimerization mutations. Fab 1 and 2 contain differential S-S bridges that ensure correct LC and HC pairing.

In some embodiments, the multi-specific binding protein is in an CrossmAb format which is an heterodimeric construct with 2 different Fabs binding to Target 1 and 2 fused to Fc stabilized by heterodimerization. CL and CH1 domains and VH and VL domains are switched, e.g. CH1 is fused in-line with VL, while CL is fused in-line with VH.

In some embodiments, the multi-specific binding protein is in an CrossmAb format which is an homodimeric constructs where Fab binding to antigen 2 is fused to the N terminus of HC of Fab that binds to antigen 1.

### Heterodimeric Antibody Heavy Chains

Assembly of heterodimeric antibody heavy chains can be accomplished by expressing two different antibody heavy chain sequences in the same cell, which may lead to the assembly of homodimers of each antibody heavy chain as well as assembly of heterodimers. Preferential assembly of heterodimeric heavy chains within the multi-specific binding proteins described herein can be promoted by incorporating distinct pairs of amino acid substitutions into the first CH3 domain within the first heavy chain polypeptide and the second CH3 domain within the second heavy chain polypeptide that allow these two chains to selectively heterodimerize with each other, as shown in U.S. Ser. Nos. 13/494,870, 16/028,850, 11/533,709, 12/875,015, 13/289,934, 14/773,418, 12/811,207, 13/866,756, 14/647,480, 14/830,336. In some embodiments, the multi-specific binding proteins contain the Fc domain of human IgG1. Various examples of amino acid substitutions within the pair of human IgG1 Fc domains are listed below to facilitate heterodimerization of two heavy chains. Each positions of amino acid substitutions is numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine(S), threonine (T), or valine (V), such that the larger amino acid substitution (a protuberance) fits into the surface of the smaller amino acid substitutions (a cavity). For example, one polypeptide can include a T366W substitution, and the other can include three substitutions including T366S, L368A, and Y407V.

Alternatively, amino acid substitutions could be selected from the following sets of substitutions shown in Table 2.

TABLE 2

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 3.

TABLE 3

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 4.

TABLE 4

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 5.

TABLE 5

| First Polypeptide | Second Polypeptide |
|---|---|
| L351Y, D399R, D399K, | T366V, T366I, T366L, T366M, N390D, |
| S400K, S400R, Y407A, | N390E, K392L, K392M, K392V, K392F |
| Y407I, Y407V | K392D, K392E, K409F, K409W, T411D |
| | and T411E |

Alternatively, at least one amino acid substitutions could be selected from the following set of substitutions in Table 6, where the position(s) indicated in the First Polypeptide column is replaced by any known negatively-charged amino acid, and the position(s) indicated in the Second Polypeptide Column is replaced by any known positively-charged amino acid.

TABLE 6

| First Polypeptide | Second Polypeptide |
|---|---|
| K392, K370, K409, or K439 | D399, E356, or E357 |

Alternatively, at least one amino acid substitutions could be selected from the following set of in Table 7, where the position(s) indicated in the First Polypeptide column is replaced by any known positively-charged amino acid, and the position(s) indicated in the Second Polypeptide Column is replaced by any known negatively-charged amino acid.

TABLE 7

| First Polypeptide | Second Polypeptide |
|---|---|
| D399, E356, or E357 | K409, K439, K370, or K392 |

Alternatively, amino acid substitutions could be selected from the following set of in Table 8.

TABLE 8

| First Polypeptide | Second Polypeptide |
|---|---|
| T350V, L351Y, F405A, and | T350V, T366L, K392L, and |
| Y407V | T394W |

Alternatively, or in addition, the structural stability of heterodimeric heavy chains within the multi-specific binding proteins can be increased by introducing S354C on either of the first or second polypeptide chain, and Y349C on the opposing polypeptide chain, which forms an artificial disulfide bridge within the interface of the two polypeptides.

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the multi-specific binding proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### A Protein Comprising an Antigen-Binding Site that Competes with the NKG2D-Binding Sites Described Herein

In certain embodiments, the present invention provides a protein that includes an antigen-binding site that competes with the NKG2D-binding sites described herein to bind to NKG2D. The NKG2D-binding sites described herein comprises amino acid sequences of SEQ ID NOs: 1 and 2; amino acid sequences of SEQ ID NOs: 3 and 4; amino acid sequences of SEQ ID NOs: 5 and 6; amino acid sequences of SEQ ID NOs: 7 and 8; amino acid sequences of SEQ ID NOs: 9 and 10; amino acid sequences of SEQ ID NOs: 83 and 8; amino acid sequences of SEQ ID NOs: 84 and 8; amino acid sequences of SEQ ID NOs: 85 and 8; amino acid sequences of SEQ ID NOs: 86 and 8; or amino acid sequences of SEQ ID NOs: 87 and 8. These NKG2D-binding sites can bind to different epitopes on NKG2D mapped by surface plasmon resonance. For example, ADI-27744 binds to a different epitope on NKG2D from ADI-27749 and other existing NKG2D antibodies, as shown in Example 2.

In some embodiments, an antigen-binding site of the protein that competes with the NKG2D-binding sites includes a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO: 1 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:2. In some embodiments, an antigen-binding site of the protein that competes with the NKG2D-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO: 3 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:4. In some embodiments, an antigen-binding site of the protein that competes with the NKG2D-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:5 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:6. In some embodiments, an antigen-binding site of the protein that competes with the NKG2D-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:7 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO: 8. In some embodiments an antigen-binding site of the protein that competes with the NKG2D-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:9 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:10.

In some embodiments, the protein that includes an antigen-binding site that competes with NKG2D-binding sites described herein further includes a second antigen-binding site that binds a tumor-associated antigen and/or a CD16 binding site. In some embodiments, the CD16 binding site is an antibody constant region or a portion thereof capable of binding CD16. In some embodiments, the CD16 binding site contains a human IgG1 Fc domain.

### Cell for Expressing a Protein

In one aspect, the present disclosure provides a cell comprising one or more nucleic acids encoding a protein that contains: an NKG2D-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2; a NKG2D-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:3, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:4; an NKG2D-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO: 6; an NKG2D-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:7, 83, 84, 85, 86, or 87, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:8; or an NKG2D-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO: 10.

### Therapeutic Applications

The invention provides methods for enhancing tumor cell death and/or treating cancer using a multi-specific binding protein described herein and/or a pharmaceutical composition described herein. The methods may be used to treat a variety of cancers. The type of cancer to be treated is desirably matched with the type of cancer cell to which the protein binds. Additional aspects and embodiments of the therapeutic methods are described below.

### Pharmaceutical Compositions

In one aspect, the present disclosure also features pharmaceutical compositions that contain an effective amount of a protein, which contains an NKG2D-binding site described herein or an NKG2D-binding site that competes with the NKG2D-binding sites described herein, and a pharmaceutically acceptable carrier.

In certain embodiments, the formulation includes a protein that comprises an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO: 1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:3, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:4. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:7, 83, 84, 85, 86, or 87, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:8. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g. 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation includes an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g.

mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/ml of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a mass decrease of 17 Da from the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg-about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lycoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution. In certain embodiments, the lyophilized drug product of the current disclosure is reconstituted with cither Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308:43-53, 2001; Steimer et al., Clinica Chimica Acta 308:33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### Enhancing Tumor Cell Death and Cancer Treatment

The invention provides methods of enhancing tumor cell death and/or treating cancer in patient. In some embodiments, the method comprises exposing a tumor and natural killer cells to a multi-specific binding protein disclosed herein. In some embodiments, the method includes administering to a patient in need thereof a therapeutically effective amount of a protein and or its desired formulation described herein. In those embodiments, the multi-specific binding protein can contain: an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2; an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO: 3 and a light chain variable domain having an amino acid sequence at least 90% (e.g., 91%, 91%, 93%, 94%, 95%, 96%, 97%, 98%, 99%, or 100%) identical to the amino acid sequence of SEQ ID NO:4; an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5 and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6; an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO: 7, 83, 84, 85, 86, or 87 and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:8; or an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9 and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10.

The type of cancer to be treated is desirably matched with the type of cancer cell to which the multi-specific binding protein disclosed herein binds. For example, treatment of a cancer expressing epithelial cell adhesion molecule (EpCAM), such as a colon cancer expressing EpCAM, is desirably treated using a multispecific-binding protein described herein that binds to EpCAM and NKG2D.

In some embodiment, patients to be treated contain cancer cells that expresses one or more of the following: CD33, HER2, CD2, CD19, CD20, CD30, CD38, CD40, CD52, CD70, EGFR/ERBB1, IGF1R, HER3/ERBB3, HER4/ERBB4, MUC1, CEA, cMET, SLAMF7, PSCA, MICA, MICB, TRAILR1, TRAILR2, MAGE-A3, B7.1, B7.2, CTLA4, and PD1. In some embodiments, the patients to be treated have a solid cancer, such as brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In some embodiments, the patients to be treated have non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In some embodiments, proteins described herein are used in combination with additional therapeutic agents to treat patients with cancer. Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein described herein may be administered during a time when the additional therapeutic agent(s) exerts its prophylactic or therapeutic effect, or vice versa.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1—Binding Affinities of Various NKG2D-Binding Domains

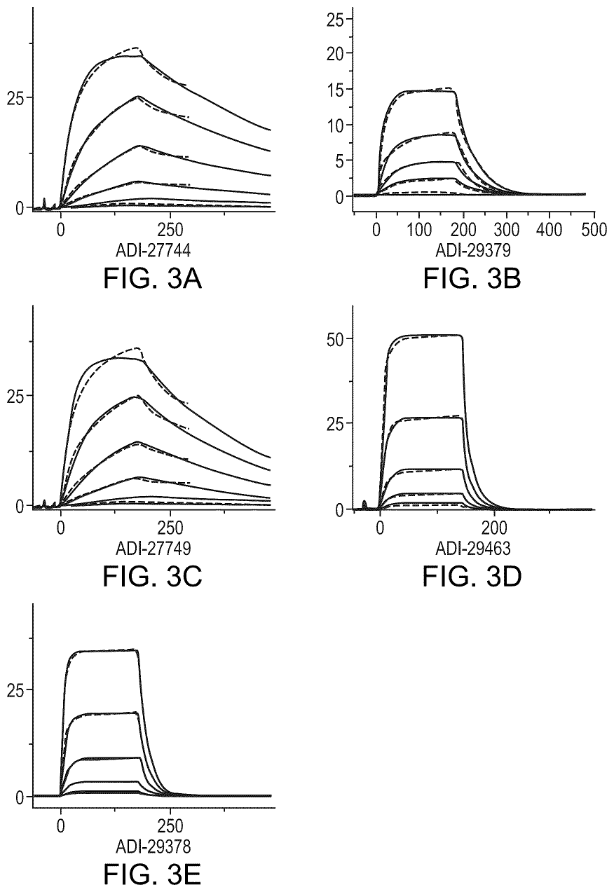

Kinetics and affinity of various NKG2D-binding domains were assessed by surface plasmon resonance using Biacore 8K instrument (GE Healthcare). Anti-human Fc antibody was immobilized on a CM5 chip using standard amine coupling chemistry. Human monoclonal antibodies containing various NKG2D-binding domains were captured on the anti-human Fc chip at a density of approximately 100 RU. Solutions containing 0.411-100 nM soluble mouse Fc-human NKG2D dimers were injected over the captured NKG2D antibodies and control surfaces at 30 μl/min at 37° C. Surfaces were regenerated between cycles by quick injection of 10 mM glycine, pH 1.8. To obtain kinetic rate constants, double-referenced data were fit to a 1:1 interaction model using Biacore 8K Evaluation software (GE Healthcare). The equilibrium binding constant K_{D} was determined by the ratio of dissociation constant k_{d} and association constant kₐ (k_{d}/kₐ). As shown in Table 9 and FIG. 3, binding affinities of NKG2D-binding domains to NKG2D are in the range of 10-62 nM.

TABLE 9

| NKG2D-binding domain | kₐ (1/Ms) | k_{d} (1/s) | K_{D} (nM) |
|---|---|---|---|
| ADI-27744 (A44) | 2.95E+05 | 2.99E−03 | 10.1 |
| ADI-27749 (A49) | 3.95E+05 | 4.89E−03 | 12.4 |
| ADI-29378 (E78) | 8.32E+05 | 4.87E−02 | 58.5 |
| ADI-29379 (E79) | 4.43E+05 | 2.25E−02 | 50.7 |
| ADI-29463 (F63) | 1.64E+06 | 1.01E−01 | 61.8 |

### Example 2—Binding Epitope Binning of ADI-27744 Clone

The binning of ADI-27744 (A44) NKG2D-binding domain was performed against a series of antibodies and ULBP6 (NKG2D natural ligand) by surface plasmon resonance using a Biacore 8K instrument. Briefly, mouse Fc-human NKG2D was captured using an anti-mouse Fc antibody immobilized on a CM5 chip at a density of approximately 100 RU. This was followed by consecutive injections of antibodies, including an NKG2D monoclonal antibody comprising ADI-27744, ADI-27749, F47 (sequences listed below) or 1D11 (a commercial monoclonal NKG2D antibody), ULBP6 (sequence listed below), MS (NKG2D antibody from Novo Nordisk, sequences listed below), and MAB139 (NKG2D antibody from R&D system, clone 149810) at 30 μl/min at 25° C. Biacore 8K evaluation software was used for all data analysis.

TABLE 10

| | Heavy chain variable region | Light chain variable region |
|---|---|---|
| F47 | QVQLQQWGAGLLKPSETLSLTCAVY | DIQMTQSPSTLSASVGDRVTITCRA |
| | GGSFSGYYWSWIRQPPGKGLEWIGEI | SQSISSWLAWYQQKPGKAPKLLIY |
| | DHSGSTNYNPSLKSRVTISVDTSKNQ | KASSLESGVPSRFSGSGSGTEFTLTI |
| | FSLKLSSVTAADTAVYYCARARGPW | SSLQPDDFATYYCQQYDTFITFGGG |
| | SFDPWGQGTLVTVSS | TKVEIK |
| | (SEQ ID NO: 51) | (SEQ ID NO: 55) |
| | CDR1 (SEQ ID NO: 52) - GSFSGYYWS | CDR1 (SEQ ID NO: 56) - |
| | CDR2 (SEQ ID NO: 53) - | RASQSISSWLA |
| | EIDHSGSTNYNPSLKS | CDR2 (SEQ ID NO: 57) - KASSLES |
| | CDR3 (SEQ ID NO: 54) - | CDR3 (SEQ ID NO: 58) - QQYDTFIT |
| | ARARGPWSFDP | |
| MS | QVHLQESGPGLVKPSETLSLTCTVSD | EIVLTQSPGTLSLSPGERATLSCRAS |
| | DSISSYYWSWIRQPPGKGLEWIGHIS | QSVSSSYLAWYQQKPGQAPRLLIY |
| | YSGSANYNPSLKSRVTISVDTSKNQF | GASSRATGIPDRESGSGSGTDFTLTI |
| | SLKLSSVTAADTAVYYCANWDDAF | SRLEPEDFAVYYCQQYGSSPWTFG |
| | NIWGQGTMVTVSS | GGTKVEIK |
| | (SEQ ID NO: 59) | (SEQ ID NO: 63) |
| | CDR1 (SEQ ID NO: 60) - SYYWS | CDR1 (SEQ ID NO: 64) - |
| | CDR2 (SEQ ID NO: 61) - | RASQSVSSSYLA |
| | HISYSGSANYNPSLKS | CDR2 (SEQ ID NO: 65) - GASSRAT |
| | CDR3 (SEQ ID NO: 62) - WDDAFNI | CDR3 (SEQ ID NO: 66) - |
| | | QQYGSSPWT |

RRDDPHSLCYDITVIPKFRPGPRWCAVQGQVDEKTFLHYDCGNKTVTPVS
PLGKKLNVTMAWKAQNPVLREVVDILTEQLLDIQLENYTPKEPLTLQARM
SCEQKAEGHSSGSWQFSIDGQTFLLFDSEKRMWTTVHPGARKMKEKWEND
KDVAMSFHYISMGDCIGWLEDFLMGMDSTLEPSAGAPLAMSSG

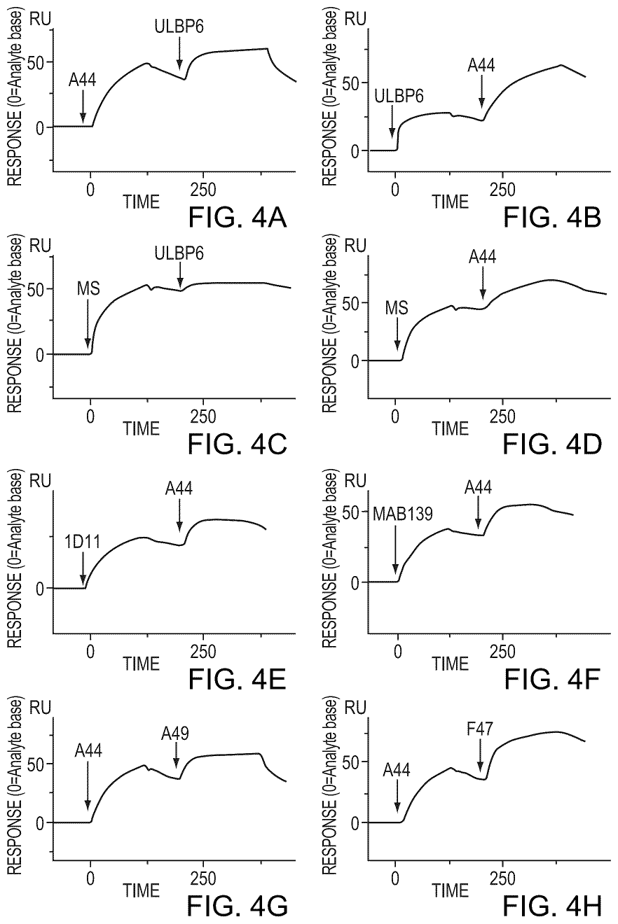

FIG. 4A shows the profile that an NKG2D monoclonal antibody comprising an ADI-27744 was injected over the immobilized NKG2D, followed by injection of ULBP6. FIG. 4B shows the profile of ULBP6 that was injected over an the immobilized NKG2D, followed by injection of NKG2D monoclonal antibody including ADI-27744. These results show that NKG2D monoclonal antibody including an ADI-27744 antigen-binding site does not block ULBP6 binding to NKG2D, i.e., ADI-27744 binds to an different epitope on NKG2D from ULBP6.

FIG. 4C shows the profile that MS monoclonal antibody was injected over the NKG2D, followed by injection of ULBP6. MS monoclonal antibody blocks ULBP6 from binding to NKG2D. FIGS. 4D-F shows the profile that MS, 1D11, or MAB139 was injected over the immobilized NKG2D, followed by injection of NKG2D monoclonal antibody comprising ADI-27744. FIGS. 4G-H shows the profile that NKG2D monoclonal antibody comprising ADI-27744 was injected over immobilized NKG2D, followed by injection of NKG2D monoclonal antibody comprising ADI-27749 or F47. ADI-27744 does not block the binding of MS, 1D11, and MAB139 to NKG2D. ADI-27749 and F47 do not block the binding of ADI-27744 to NKG2D. These results indicate that ADI-27744 binds to a unique epitope on NKG2D, different from the MS, 1D11, MAB139, ADI-27749 and F47 binding epitope(s).

### Example 3—Trispecific Binding Proteins Bind to NKG2D

EL4 mouse lymphoma cell lines were engineered to express human NKG2D. Trispecific binding proteins (TriNKETs) that each contain an NKG2D-binding domain, a tumor-associated antigen-binding domain (such as a CD33 or a HER2-binding domain), and an Fc domain that binds to CD16 as shown in FIG. 1, were tested for their affinity to extracellular NKG2D expressed on EL4 cells. The binding of the multispecific binding proteins to NKG2D was detected using fluorophore-conjugated anti-human IgG secondary antibodies. Cells were analyzed by flow cytometry, and fold-over-background (FOB) was calculated using the mean fluorescence intensity (MFI) of NKG2D expressing cells compared to parental EL4 cells.

TriNKETs tested include CD33-TriNKET-A44 (ADI-27744 and a CD33 binding domain), CD33-TriNKET-A49 (ADI-27749 and a CD33 binding domain), CD33-TriNKET-F63 (ADI-29463 and a CD33 binding domain), HER2-TriNKET-A44 (ADI-27744 and a CD33 binding domain), HER2-TriNKET-A49 (ADI-27749 and a HER2 binding domain), HER2-TriNKET-F63 (ADI-29463 and a HER-binding domain), and HER2-TriNKET-E79 (ADI-29379 and a HER2 binding domain). The HER2-binding domain is composed of a heavy chain variable domain and a light chain variable domain of Trastuzumab. The CD33 binding domain is composed of a heavy chain variable domain and a light chain variable domain listed below.

```
SEQ ID NO: 49
QVQLVQSGAEVKKPGASVKVSCKASGYTFTDYVVHWVRQAPGQGLEWMG
                                                              CDR1
YINPYNDGTKYNEKFKGRVTMTRDTSISTAYMELSRLRSDDTAVYYCAR
                  CDR2
DYRYEVYGMDYWGQGTLVTVSS
      CDR3
SEQ ID NO: 50
DIVLTQSPASLAVSPGQRATITCTASSSVNYIHWYQQKPGQPPKLLIY
                                                    CDR1
DTSKVASGVPARFSGSGSGTDFTLTINPVEANDTANYYCQQWRSYPLT
  CDR1 CDR3
FGQGTKLEIK
```

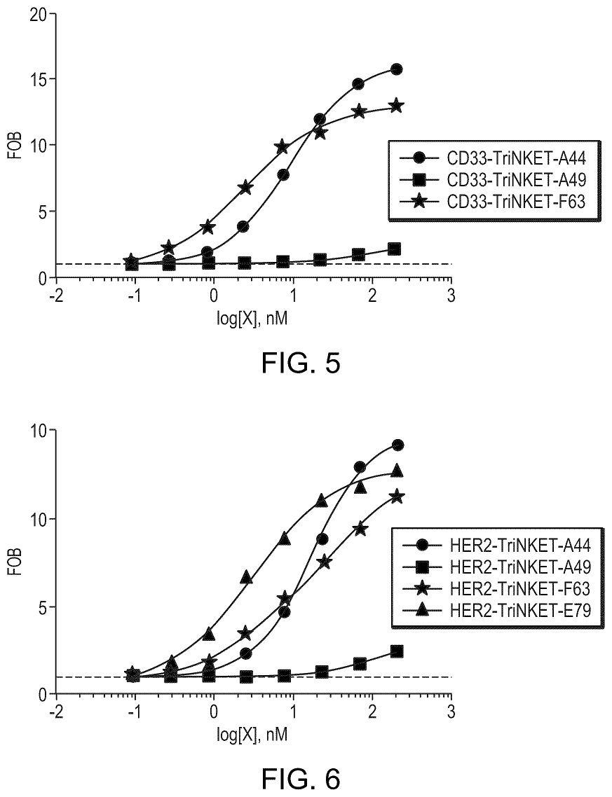

All TriNKETs bind NKG2D on EL4 cells, but with different affinities. CD33-TriNKET-A44 show the same binding profile as HER2-TriNKET-A44, so do CD33-TriNKET-A49 as HER2-TriNKET-A49, and CD33-TriNKET-F63 to HER2-TriNKET-F63. The NKG2D-binding affinity for each clone was similar between cells expressing human and mouse NKG2D (FIGS. 5-6).

### Example 4—Trispecific Binding Proteins Bind to Human Tumor Antigens

### Trispecific Binding Proteins Bind to CD33

Human AML cell line MV4-11, expressing CD33 was used to assay the binding of TriNKETs to the tumor-associated antigen. TriNKETs and the parental CD33 monoclonal antibody were incubated with the cells, and the binding was detected using fluorophore-conjugated anti-human IgG secondary antibodies. Cells were analyzed by flow cytometry, and fold-over-background (FOB) was calculated using the mean fluorescence intensity (MFI) from TriNKETs and the parental monoclonal CD33 antibody normalized to secondary antibody controls.

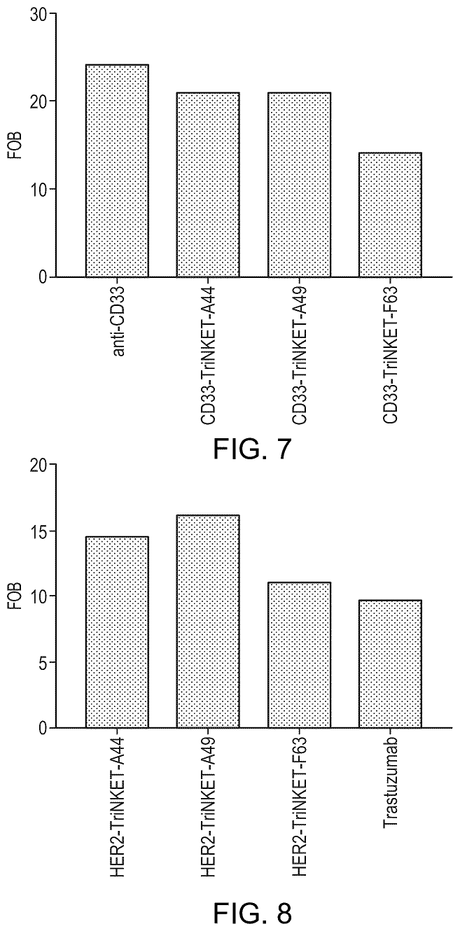

CD33-TriNKET-A44, CD33-TriNKET-A49, and CD33-TriNKET-F63 show comparable levels of binding to CD33 as compared with the parental CD33 antibody (FIG. 7).

### Trispecific Binding Proteins Bind to HER2

Human cancer cell lines expressing HER2 were used to assay the binding of TriNKETs to the tumor-associated antigen. Renal cell carcinoma cell line 786-O expresses low level of HER2, and human lung cancer cell line NCI-H661 expresses moderate levels of HER2. TriNKETs and optionally the parental HER2 monoclonal antibody (Trastuzumab) were incubated with the cells, and the binding was detected using fluorophore-conjugated anti-human IgG secondary antibodies. Cells were analyzed by flow cytometry, and fold-over-background (FOB) was calculated using the mean fluorescence intensity (MFI) from TriNKETs and Trastuzumab normalized to secondary antibody controls.

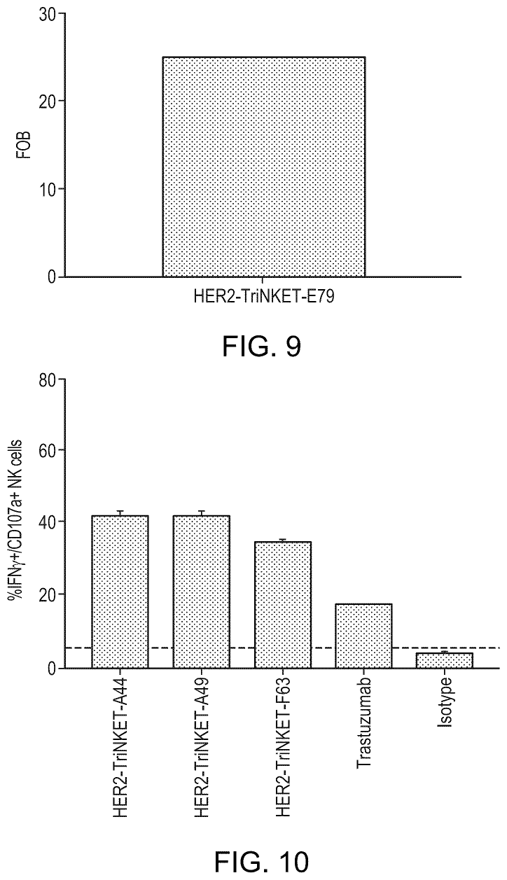

HER2-TriNKET-A44, HER2-TriNKET-A49, and HER2-TriNKET-F63 show comparable levels of binding to HER2 expressed on 786-O cells as compared with Trastuzumab (FIG. 8). Binding to HER2 expressed on NCI-H661 cells by HER2-TriNKET-E79 is shown (FIG. 9).

### Example 5—Trispecific Binding Proteins Activate NK Cells

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >90%. Isolated NK cells were cultured in media containing 100 ng/ml IL-2 for activation or rested overnight without cytokine. IL-2-activated NK cells were used within 24-48 hours after activation.

Human cancer cells expressing a tumor antigen were harvested and resuspended in culture media at 2×10⁶ cells/mL. Monoclonal antibodies or TriNKETs targeting the tumor antigen were diluted in culture media. Activated NK cells were harvested, washed, and resuspended at 2×10⁶ cells/mL in culture media. Cancer cells were then mixed with monoclonal antibodies/TriNKETs and activated NK cells in the presence of IL-2. Brefeldin-A and monensin were also added to the mixed culture to block protein transport out of the cell for intracellular cytokine staining. Fluorophore-conjugated anti-CD107a was added to the mixed culture and the culture was incubated for 4 hrs before samples were prepared for FACS analysis using fluorophore-conjugated antibodies against CD3, CD56 and IFN-gamma. CD107a and IFN-gamma staining was analyzed in CD3⁻CD56⁺ cells to assess NK cell activation. The increase in

CD107a/IFN-gamma double-positive cells is indicative of better NK cell activation through engagement of two activating receptors rather than one receptor.

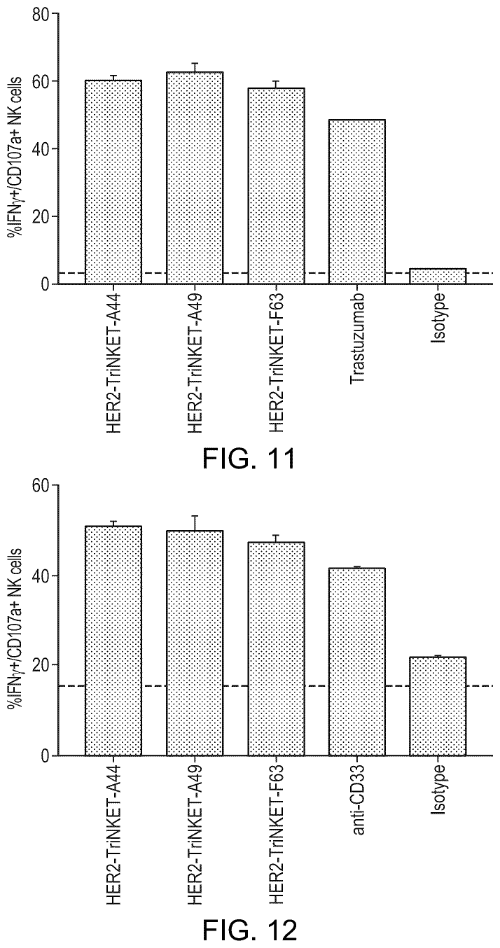

TriNKETs mediate activation of human NK cells co-cultured with HER2-expressing NCI-H661 cells (FIG. 10) and SkBr-3 cells (FIG. 11) respectively as indicated by an increase of CD107a degranulation and IFN-gamma production. Compared to the monoclonal antibody Trastuzumab, TriNKETs show superior activation of human NK cells in the presence of human cancer cells.

TriNKETs mediate activation of human NK cells co-cultured with CD33-expressing human AML Mv4-11 cells as shown by an increase of CD107a degranulation and IFN-gamma production (FIG. 12). Compared to the monoclonal anti-CD33 antibody, TriNKETs show superior activation of human NK cells in the presence of human cancer cells.

### Example 6—Trispecific Binding Proteins Enable Cytotoxicity of Target Cancer Cells

Peripheral blood mononuclear cells (PBMCs) were isolated from human peripheral blood buffy coats using density gradient centrifugation. NK cells (CD3⁻CD56+) were isolated using negative selection with magnetic beads from PBMCs, and the purity of the isolated NK cells was typically >90%. Isolated NK cells were cultured in media containing 100 ng/ml IL-2 for activation or rested overnight without cytokine. IL-2-activated or rested NK cells were used the following day in cytotoxicity assays.

In order to test the ability of human NK cells to lyse cancer cells in the presence of TriNKETs, a cytoTox 96 non-radioactive cytotoxicity assay from Promega (G1780) was used according to manufacturer's instruction. Briefly, human cancer cells expressing a tumor antigen were harvested, washed, and resuspended in culture media at 1-2×10⁵ cells/mL. Rested and/or activated NK cells were harvested, washed, and resuspended at 10⁵-2.0×10⁶ cells/mL in the same culture media as that of the cancer cells. In each well of a 96 well plate, 50 μl of the cancer cell suspension was mixed with 50 μl of NK cell suspension with or without TriNKETs targeting the tumor antigen expressed on the cancer cells. After incubation at 37° C. with 5% CO₂ for 3 hours and 15 minutes, 10× lysis buffer was added to wells containing only cancer cells, and to wells containing only media for the maximum lysis and negative reagent control respectively. The plate was then placed back into the incubator for an additional 45 minutes to reach a total of 4 hours incubation. Cells were then pelleted, and the culture supernatant was transferred to a new 96 well plate and mixed with a substrate for development. The new plate was incubated for 30 minutes at room temperature, and the absorbance was read at 492 nm on a SpectraMax i3x. Percentage of specific lysis of the cancer cells was calculated as follows: % Specific lysis=((experimental lysis−spontaneous lysis from NK cells alone−spontaneous lysis from cancer cells alone)/(Maximum lysis−negative reagent control))*100%

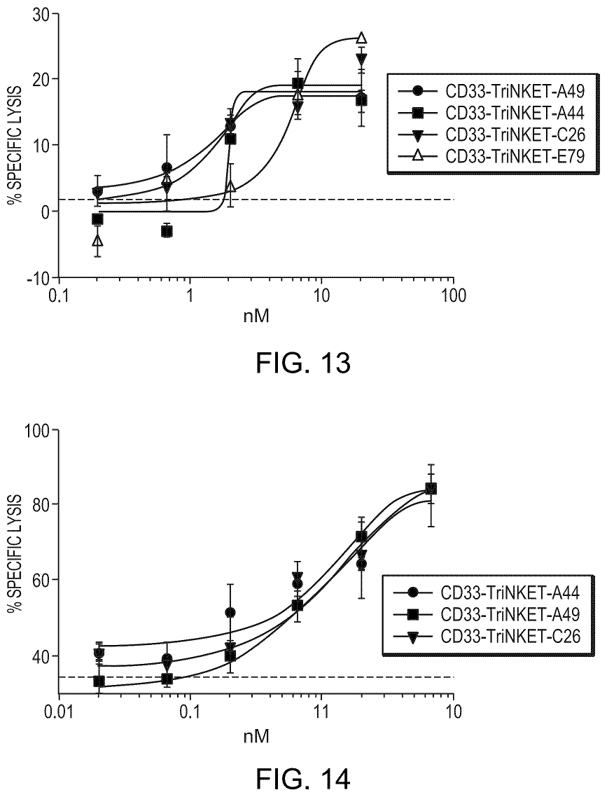

TriNKETs mediate cytotoxicity of human NK cells against the CD33-positive Molm-13 human AML cell line. As shown in FIG. 13, rested human NK cells were mixed with Molm-13 cancer cells, and TriNKETs are able to enhance the cytotoxic activity of rested human NK cells in a dose-responsive manor against the cancer cells. The dotted line indicates cytotoxic activity of rested NK cells without TriNKETs. As shown in FIG. 14, activated human NK cells were mixed with Molm-13 cancer cells, and TriNKETs enhance the cytotoxic activity of activated human NK cells even further in a dose-responsive manor against the cancer cells.

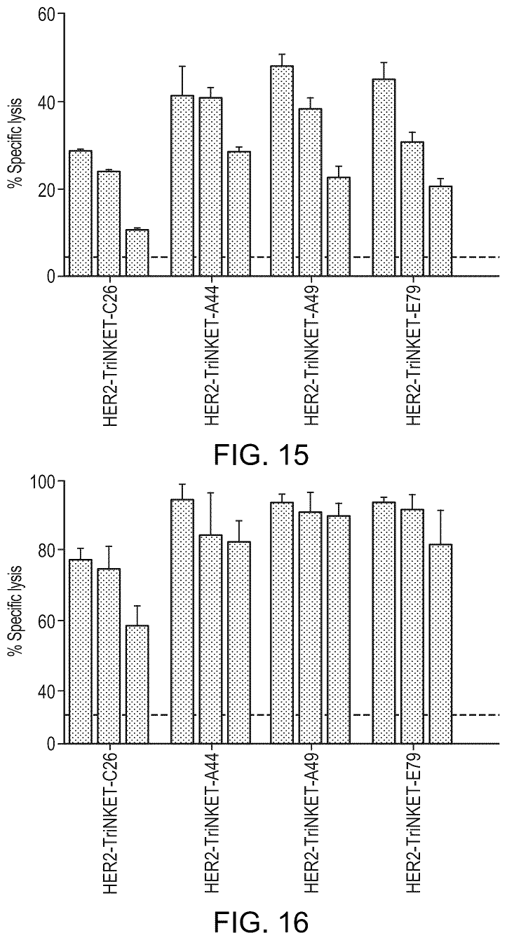

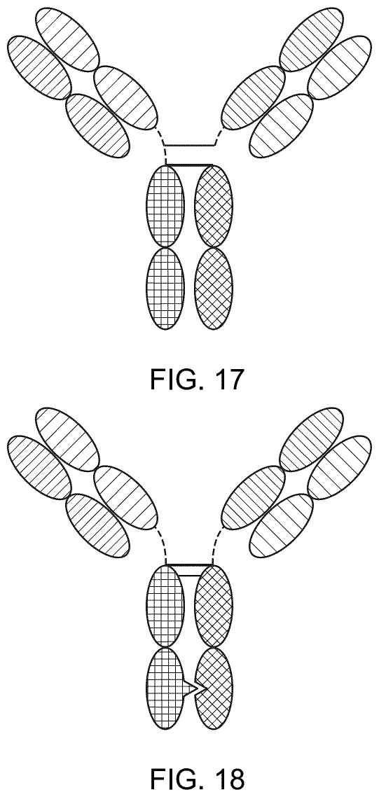

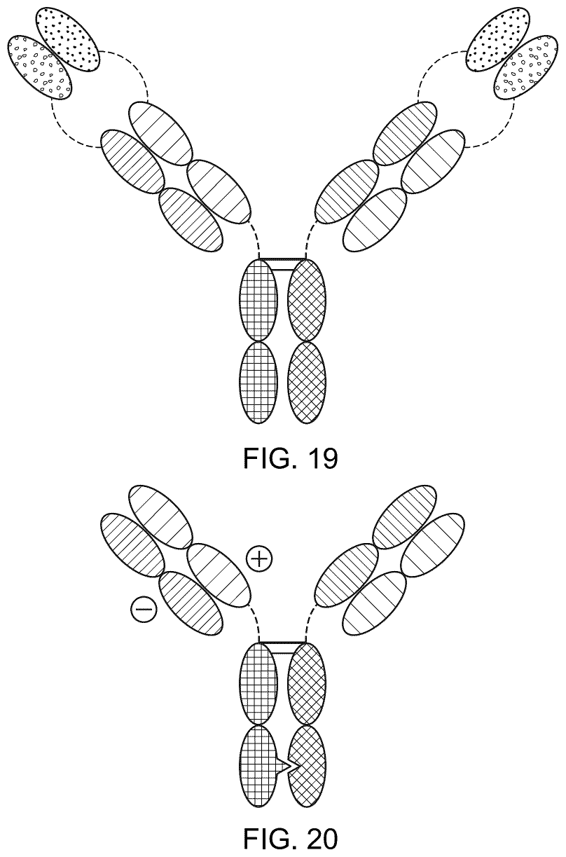

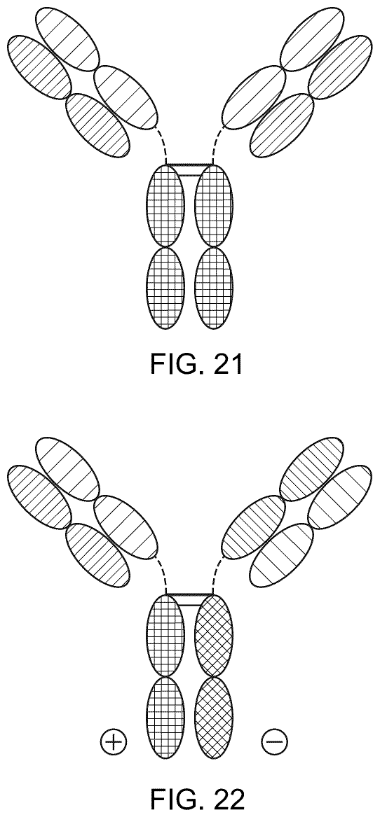

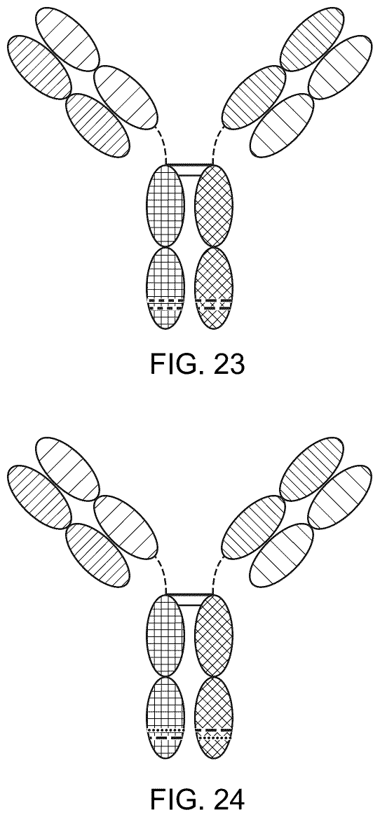

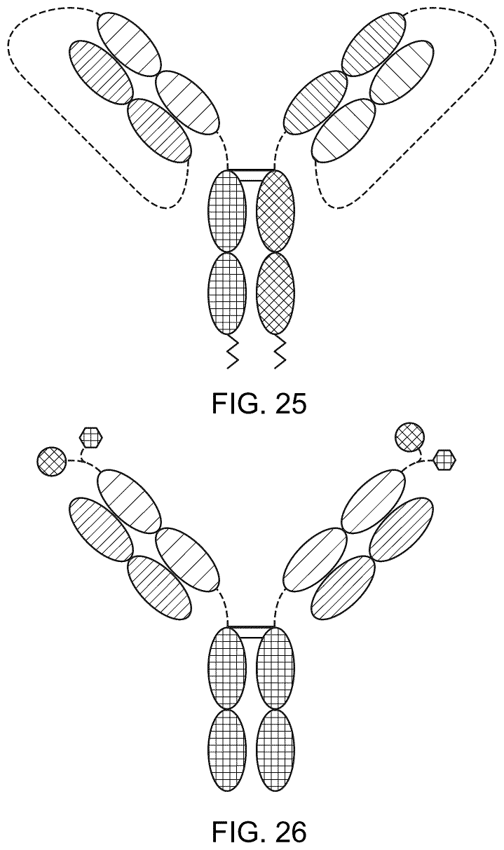

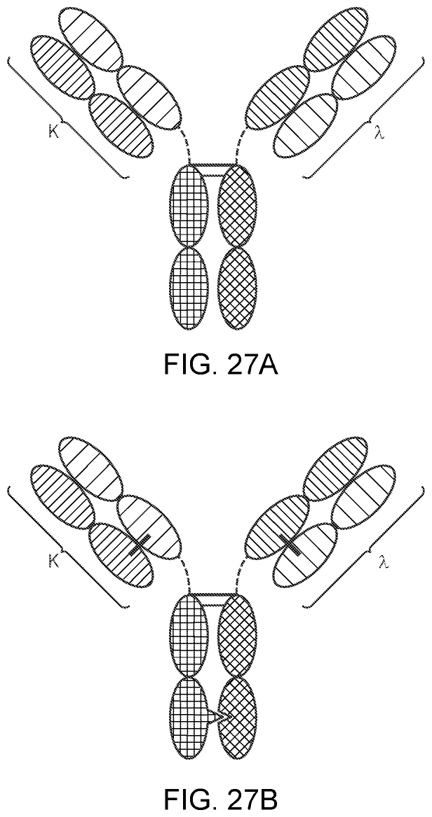

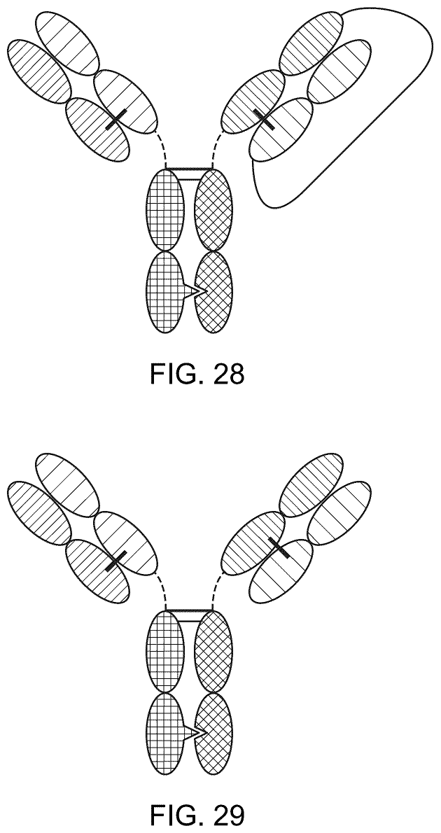

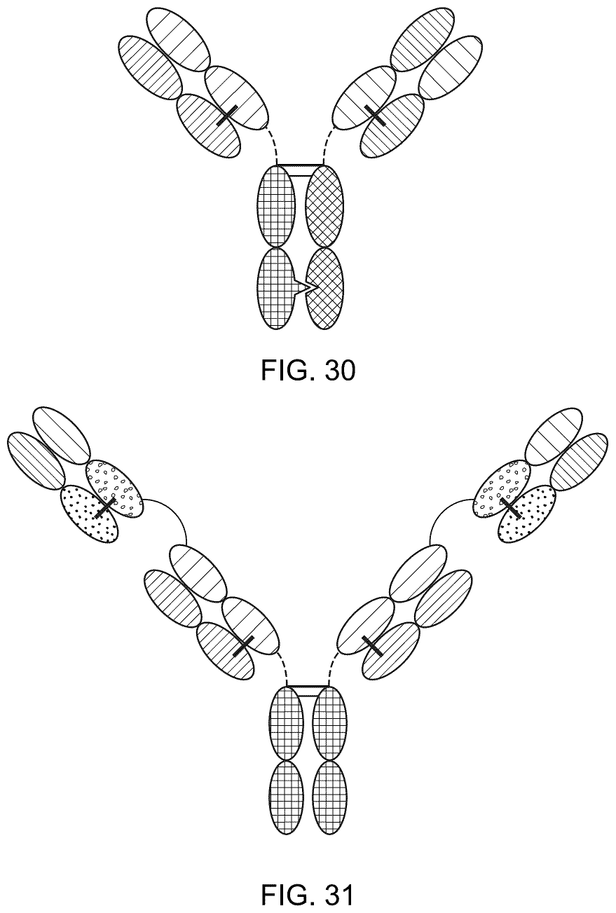

TriNKETs mediate cytotoxicity of human NK cells against the HER2-positive 786-O human renal cell carcinoma cell line. As shown in FIG. 15, rested human NK cells were mixed with 786-O cancer cells, and TriNKETs are able to enhance the cytotoxic activity of rested human NK cells in a dose-responsive manor against the cancer cells (each TriNKET was added at 5, 1, 0.2 μg/ml in the assay and the results are represented in 3 columns from the left to the right in each TriNKET in the FIGS. 15-16). Dotted line indicates the cytotoxic activity of rested NK cells against 786-O cells in the absence of TriNKETs. As shown in FIG. 16, activated human NK cells were mixed with 786-O cells, and TriNKETs enhance the cytotoxic activity of activated human NK cells even further in a dose-responsive manor against the cancer cells. Dotted line indicates the cytotoxic activity of activated NK cells against 786-O cells in the absence of TriNKETs.

### Example 7—Variants of ADI-27749 and TriNKETs Containing the Variants

As described above, ADI-27749 (A49) contains, inter alia, a heavy chain CDR3 having the amino acid sequence of GAPMGAAAGWFDP (SEQ ID NO:71). The Met at position 102 of SEQ ID NO:7 (i.e., at position 4 of this CDR3 sequence) may be replaced by Gln, Leu, Ile, Phe, or Val, thereby generating NKG2D antibodies A49MQ, A49ML, A49MI, A49MF, and A49MV, respectively, having the corresponding heavy chain variable region, light chain variable region, and CDR sequences provided in Table 1.

The effects of these mutations on hydrophobicity were analyzed using the MOE2018.01 program using the parameter setting of avg_pro_patch_cdr_hyd. Residues were mutated using the protein builder module and entire Fab was minimized after tethering all residues. Dynamic property sampling was performed using the lowMD protocol in BIOMOE. As shown in Table 11, these mutations did not have a substantial negative effect on the predicted hydrophobicity of the A49 Fab.

TABLE 11

| Amino acid residue | avg_pro_patch_cdr_hyd |
|---|---|
| M | 524.0968 |
| L | 529.67743 |
| I | 551.93549 |
| V | 477.09677 |
| Q | 447.09677 |
| F | 542.25806 |

The hydrophobicity of a TriNKET containing A49 (“TriNKET A”) and a mutant form of TriNKET A having a substitution of Ile, Leu, Val, Gln, or Phe for the Met (“TriNKET A*”) were tested by analytical hydrophobic interaction chromatography (HIC). Each of the TriNKETs also bound to a first tumor antigen. As shown in Table 12, the retention time of TriNKET A* was similar to that of TriNKET A.

TABLE 12

| Protein | Retention time |
|---|---|
| TriNKET A* | 8.6 min |
| TriNKET A | 8.65 ± 0.05 min |

Thermal stability of TriNKET A and TriNKET A* was examined by differential scanning calorimetry analysis (DSC) in 20 mM Histidine, 260 mM sucrose, and 0.005% PS-80 at pH 6.0. The values of the Tₘ are shown in Table 13, where Tₘ is the midpoint transition temperature of an individual domain. The M102 mutation had a small effect on the Tₘ values of the two most stable transitions (Tₘ₃ and Tₘ₄) by shifting them 0.6 and 0.7° C. lower, compared to the TriNKET A. The earlier transitions (Tₘ₁ and Tₘ₂) were unaffected. Therefore, the M102 mutation had only a marginal effect on the overall thermal stability of TriNKET A.

TABLE 13

| Protein | Tₘ1 | Tₘ2 | Tₘ3 | Tₘ4 |
|---|---|---|---|---|
| TriNKET A | 66.2 | 80.2 | 86.3 | 88.4 |
| TriNKET A* | 66.2 | 80.5 | 85.7 | 87.7 |

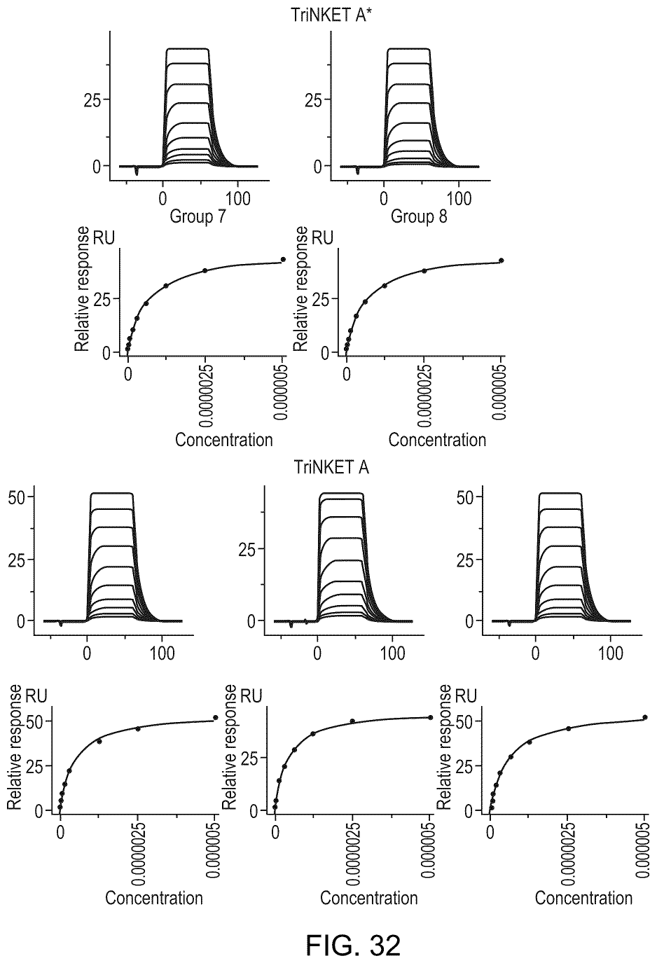

Binding of TriNKET A and TriNKET A* to a fusion protein of human NKG2D and murine Fc (“mFc-hNKG2D”) was characterized by surface plasmon resonance (SPR) at 37° C. Two different fits, steady state affinity fit and kinetic fit, were utilized to obtain the equilibrium affinity data (FIG. 32). The kinetic constants and equilibrium affinity constants were calculated, and data from the two independent experiments for TriNKET A* and the three independent experiments for TriNKET A were averaged.

TABLE 14

| | | | | Kinetics | Steady State | |
|---|---|---|---|---|---|---|
| Capture | Analyte | kₐ (1/Ms) | k_{d} (1/s) | K_{D} (M) | Affinity K_{D} (M) | Stoichiometry |
| mFc- | TriNKET | 1.41 × 10⁵ | 1.31 × 10⁻¹ | 9.31 × 10⁻⁷ | 6.98 × 10⁻⁷ | 0.86 |
| hNKG2D | A* | | | | | |
| mFc- | TriNKET | 1.56 × 10⁵ | 1.28 × 10⁻¹ | 8.19 × 10⁻⁷ | 6.76 × 10⁻⁷ | 0.85 |
| hNKG2D | A* | | | | | |
| Average | | 1.49 × 10⁵ | 1.30 × 10⁻¹ | 8.75 × 10⁻⁷ | 6.87 × 10⁻⁷ | 0.85 |
| mFc- | TriNKET | 1.91 × 10⁵ | 1.16 × 10⁻¹ | 6.05 × 10⁻⁷ | 4.62 × 10⁻⁷ | 1.01 |
| hNKG2D | A | | | | | |
| mFc- | TriNKET | 2.03 × 10⁵ | 1.06 × 10⁻¹ | 5.23 × 10⁻⁷ | 4.20 × 10⁻⁷ | 0.88 |
| hNKG2D | A | | | | | |
| mFc- | TriNKET | 1.93 × 10⁵ | 1.15 × 10⁻¹ | 5.95 × 10⁻⁷ | 5.80 × 10⁻⁷ | 1.12 |
| hNKG2D | A | | | | | |

| | | | | | |
|---|---|---|---|---|---|
| Average ± stdev | (1.96 ± 0.06) × 10⁵ | (1.12 ± 0.06) × 10⁻¹ | (5.74 ± 0.45) × 10⁻⁷ | (4.87 ± 0.83) × 10⁻⁷ | 1.01 ± 0.11 |

As shown in Table 14, the equilibrium affinity constants (K_{D}) obtained from both the affinity and kinetic fits were very similar between the replicates, which suggested a high confidence in the measured parameters. The M102 variant has less than 2-fold reduced affinity for human NKG2D compared to TriNKET A. The K_{D} for TriNKET A* was (6.87±0.16)×10⁻⁷ M, while the K_{D} for TriNKET A was (4.87±0.83)×10⁻⁷ M (calculated from the affinity fit). Similar differences in affinities were observed when K_{D} was calculated from the kinetic fit. The stoichiometry of NKG2D binding to TriNKET A* was 0.85±0.12, similar to the 1.01±0.11 for TriNKET A, confirming that each NKG2D dimer binds to one molecule of TriNKET A *. This suggests that the M102 mutation had only a minor effect on the binding of an A49-containing TriNKET to human NKG2D.

Finally, the effect of the M102 mutation on the potency of TriNKETs was assessed in a cytotoxicity assay. Briefly, KHYG-1 cells expressing the high-affinity variant of CD16a (158V) were generated through retroviral transduction. Following transduction, cells were selected in puromycin-containing growth media to generate a selected population of KHYG-1-CD16V cells. The selected population was maintained in media containing 10 ng/ml human IL-2. To prepare the KHYG-1-CD16V cells for use as effectors in cytotoxicity assays, the cells were harvested from culture, pelleted, washed three times in culture media without IL-2, and resuspended in culture media without IL-2 and rested for 24 hours.

Human cancer cell lines expressing a target of interest were harvested from culture. The cells were washed with HBS, and were resuspended in growth media at 10⁶ cells/mL for labeling with BATDA reagent (Perkin Elmer C136-100). Manufacturer instructions were followed for labeling of the target cells. After labeling, the cells were washed three times with HBS and were resuspended at 0.5×10⁵ cells/mL in culture media. 100 μl of BATDA labeled cells were added to each well of a 96-well plate.

TriNKETs were serially diluted in culture media, and 50 μl of a diluted TriNKET was added to each well. Rested NK cells were harvested from culture, washed, and resuspended at 1.0×10⁶ cells/mL in culture media. 50 μl of NK cells were added to each well of the plate to attain a desired E:T ratio of 10:1 and to make a total of 200 μl culture volume in each well. The plate was incubated at 37° C. with 5% CO₂ for 2-3 hours.

After the culturing, the plate was removed from the incubator, and the cells were pelleted by centrifugation at 200×g for 5 minutes. 20 μl of culture supernatant was transferred to a clean microplate provided from the manufacturer. Supernatant from the labeled cells incubated alone without NK cells was used to measure spontaneous release of TDA. Supernatant from labeled cells incubated with 1% Triton-X was used to measure maximum lysis of the target cells. Supernatant from the labeled cells prior to the 2-3 hours of incubation was used to measure the background and for quality control purposes.

200 μl of room temperature europium solution (Perkin Elmer C135-100) was added to each well containing culture supernatant. The plate was protected from light and incubated on a plate shaker at 250 rpm for 15 minutes. Fluorescence was measured using a SpectraMax i3X instrument. The fluorescent levels represented lysis of the target cells. The values of % specific lysis were calculated as: % specific lysis=((Experimental release−Spontaneous release)/(Maximum release−Spontaneous release))×100%.

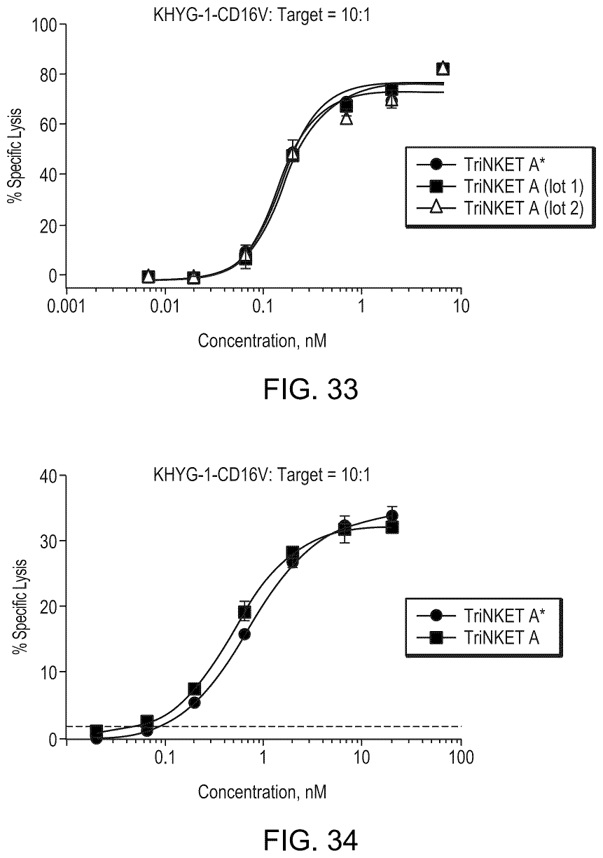

To measure the activity of TriNKET A and TriNKET A*, a cell line that expressed the first tumor antigen was selected as target cells. Two different lots of TriNKET A were used for comparison. The % specific lysis values were plotted in FIG. 33, and the EC50 and maximum % specific lysis values were summarized in Table 15. The EC50 and maximum % specific lysis values of TriNKET A* were similar to those of TriNKET A, suggesting that the M102 mutation did not affect the biological activity of TriNKET A.

TABLE 15

| Protein | EC₅₀ (nM) | Max lysis (%) |
|---|---|---|
| TriNKET A* | 0.15 | 73 |
| TriNKET A-lot 1 | 0.17 | 76 |
| TriNKET A-lot 2 | 0.15 | 76 |

To confirm that the absence of effect of the M102 mutation on TriNKET activity was not tumor antigen-specific, TriNKET A and TriNKET A* that bind to a second, different tumor antigen were constructed. The activity of the two TriNKETs were compared in cytotoxicity assays using a cell line that expressed the second tumor antigen as target cells and KHYG-1-CD16V cells as effector cells. As shown in FIG. 34, TriNKET A* demonstrated equivalent activity to TriNKET A.

### INCORPORATION BY REFERENCE

The entire disclosure of each of the patent documents and scientific articles referred to herein is included by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antibody heavy chain variable domain comprising an amino acid sequence at least 90% identical to the amino acid sequence of SEQ ID NO:7.

2. The antibody heavy chain variable domain according to claim 1, wherein the amino acid sequence comprises:
a complementarity-determining region 1 (CDR1) sequence represented by the amino acid sequence of SEQ ID NO:48;
a complementarity-determining region 2 (CDR2) sequence represented by the amino acid sequence of SEQ ID NO:30; and
a complementarity-determining region 3 (CDR3) sequence represented by the amino acid sequence of SEQ ID NO:44.

3. The antibody heavy chain variable domain according to claim 1, wherein the amino acid sequence comprises:
a CDR1 sequence represented by the amino acid sequence of SEQ ID NO:29;
a CDR2 sequence represented by the amino acid sequence of SEQ ID NO:30; and
a CDR3 sequence represented by the amino acid sequence of SEQ ID NO:31.

4. The antibody heavy chain variable domain according to claim 1, wherein the amino acid sequence comprises:
a CDR1 sequence represented by the amino acid sequence of SEQ ID NO:48;
a CDR2 sequence represented by the amino acid sequence of SEQ ID NO:30; and
a CDR3 sequence represented by the amino acid sequence of SEQ ID NO:71.

5. The antibody heavy chain variable domain according to claim 1, wherein the amino acid sequence comprises:
a CDR1 sequence represented by the amino acid sequence of SEQ ID NO:29;
a CDR2 sequence represented by the amino acid sequence of SEQ ID NO:30; and
a CDR3 sequence represented by the amino acid sequence of SEQ ID NO:77.

6. The antibody heavy chain variable domain according to claim 1, wherein the amino acid sequence comprises:
a CDR1 sequence represented by the amino acid sequence of SEQ ID NO:48;
a CDR2 sequence represented by the amino acid sequence of SEQ ID NO:30; and
a CDR3 sequence represented by the amino acid sequence of SEQ ID NO:78.

7. An antibody heavy chain variable domain comprising an amino acid sequence at least 90% identical to the amino acid sequence of SEQ ID NO:1.

8. The antibody heavy chain variable domain according to claim 7, wherein the amino acid sequence comprises:
a CDR1 sequence represented by the amino acid sequence of SEQ ID NO:11;
a CDR2 sequence represented by the amino acid sequence of SEQ ID NO:12; and
a CDR3 sequence represented by the amino acid sequence of SEQ ID NO:13.

9. The antibody heavy chain variable domain according to claim 7, wherein the amino acid sequence comprises:
a CDR1 sequence represented by the amino acid sequence of SEQ ID NO:45;
a CDR2 sequence represented by the amino acid sequence of SEQ ID NO: 12; and
a CDR3 sequence represented by the amino acid sequence of SEQ ID NO:68.

10. An antibody heavy chain variable domain comprising an amino acid sequence at least 90% identical to the amino acid sequence of SEQ ID NO:3.

11. The antibody heavy chain variable domain according to claim 10, wherein the amino acid sequence comprises:
a CDR1 sequence represented by the amino acid sequence of SEQ ID NO:17;
a CDR2 sequence represented by the amino acid sequence of SEQ ID NO:18; and
a CDR3 sequence represented by the amino acid sequence of SEQ ID NO:19.

12. The antibody heavy chain variable domain according to claim 10, wherein the amino acid sequence comprises:
a CDR1 sequence represented by the amino acid sequence of SEQ ID NO:46;
a CDR2 sequence represented by the amino acid sequence of SEQ ID NO: 18; and
a CDR3 sequence represented by the amino acid sequence of SEQ ID NO:69.

13. An antibody heavy chain variable domain comprising an amino acid sequence at least 90% identical to the amino acid sequence of SEQ ID NO:5.

14. The antibody heavy chain variable domain according to claim 13, wherein the amino acid sequence comprises:
a CDR1 sequence represented by the amino acid sequence of SEQ ID NO:23;
a CDR2 sequence represented by the amino acid sequence of SEQ ID NO:24; and
a CDR3 sequence represented by the amino acid sequence of SEQ ID NO:25.

15. The antibody heavy chain variable domain according to claim 13, wherein the amino acid sequence comprises:
a CDR1 sequence represented by the amino acid sequence of SEQ ID NO:47;
a CDR2 sequence represented by the amino acid sequence of SEQ ID NO:24; and
a CDR3 sequence represented by the amino acid sequence of SEQ ID NO:70.

16. An antibody heavy chain variable domain comprising an amino acid sequence at least 90% identical to the amino acid sequence of SEQ ID NO:9.

17. The antibody heavy chain variable domain according to claim 16, wherein the amino acid sequence comprises:
a CDR1 sequence represented by the amino acid sequence of SEQ ID NO:35;
a CDR2 sequence represented by the amino acid sequence of SEQ ID NO:36; and
a CDR3 sequence represented by the amino acid sequence of SEQ ID NO:37.

18. The antibody heavy chain variable domain according to claim 16, wherein the amino acid sequence comprises:
a CDR1 sequence represented by the amino acid sequence of SEQ ID NO:45;
a CDR2 sequence represented by the amino acid sequence of SEQ ID NO:36; and
a CDR3 sequence represented by the amino acid sequence of SEQ ID NO:72.

19. An antibody heavy chain comprising an antibody heavy chain variable domain according to any one of claims 1-18 and an amino acid sequence at least 90% identical to an antibody constant region.

20. The antibody heavy chain according to claim 19, wherein the antibody constant region is a human IgG constant region comprising hinge, CH2, and CH3 domains.

21. The antibody heavy chain according to claim 20, wherein the antibody constant region is a human IgG constant region further comprising a CH1 domain.

22. The antibody heavy chain according to any one of claims 19-21, wherein the antibody constant region is 90% identical to a IgG1 constant region.

23. The antibody heavy chain according to claim 22, wherein the amino acid sequence at least 90% identical to an antibody constant region differs from the amino acid sequence of an IgG1 constant region at Q347, Y349, L351, S354, E356, E357, K360, Q362, S364, T366, L368, K370, N390, K392, T394, D399, S400, D401, F405, Y407, K409, T411, or K439, or any combination thereof.

24. The antibody heavy chain according to claim 23, wherein the amino acid sequence at least 90% identical to an antibody constant region differs from the amino acid sequence of an IgG1 constant region by a substitution selected from the group consisting of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y4071, Y407V, K409F, K409W, K409D, T411D, T411E, K439D, and K439E, or any combination thereof.

25. An antigen-binding site comprising the antibody heavy chain variable domain according to any one of claims 1-6, and an antibody light chain variable domain comprising an amino acid sequence at least 90% identical to SEQ ID NO:8.

26. The antigen-binding site according to claim 25, wherein the light chain variable domain comprises a CDR1 sequence identical to the amino acid sequence of SEQ ID NO:32, a CDR2sequence identical to the amino acid sequence of SEQ ID NO:33, and a CDR3 sequence identical to the amino acid sequence of SEQ ID NO:34.

27. The antigen-binding site according to claim 25 or 26 having a K_{D} of between 2-120 nM, as measured by surface plasmon resonance.

28. An antigen-binding site comprising the antibody heavy chain variable domain according to any one of claims 7-9, and an antibody light chain variable domain comprising an amino acid sequence at least 90% identical to SEQ ID NO:2.

29. The antigen-binding site according to claim 28, wherein the light chain variable domain comprises a CDR1 sequence identical to the amino acid sequence of SEQ ID NO:14, a CDR2 sequence identical to the amino acid sequence of SEQ ID NO:15, and a CDR3 sequence identical to the amino acid sequence of SEQ ID NO:16.

30. The antigen-binding site according to claim 28 or 29 having a K_{D} of between 5-500 nM, as measured by surface plasmon resonance.

31. An antigen-binding site comprising the antibody heavy chain variable domain according to any one of claims 10-12, and an antibody light chain variable domain comprising an amino acid sequence at least 90% identical to SEQ ID NO:4.

32. The antigen-binding site according to claim 31, wherein the light chain variable domain comprises a CDR1 sequence identical to the amino acid sequence of SEQ ID NO:20, a CDR2 sequence identical to the amino acid sequence of SEQ ID NO:21, and a CDR3 sequence identical to the amino acid sequence of SEQ ID NO:22.

33. The antigen-binding site according to claim 31 or 32 having a K_{D} of between 6-600 nM, as measured by surface plasmon resonance.

34. An antigen-binding site comprising the antibody heavy chain variable domain according to any one of claims 13-15, and an antibody light chain variable domain comprising an amino acid sequence at least 90% identical to SEQ ID NO:6.

35. The antigen-binding site according to claim 34, wherein the light chain variable domain comprises a CDR1 sequence identical to the amino acid sequence of SEQ ID NO:26, a CDR2 sequence identical to the amino acid sequence of SEQ ID NO:27, and a CDR3 sequence identical to the amino acid sequence of SEQ ID NO:28.

36. The antigen-binding site according to claim 34 or 35 having a K_{D} of between 1-100 nM, as measured by surface plasmon resonance.

37. An antigen-binding site comprising the antibody heavy chain variable domain according to any one of claims 16-18, and an antibody light chain variable domain comprising an amino acid sequence at least 90% identical to SEQ ID NO:10.

38. The antigen-binding site according to claim 37, wherein the light chain variable domain comprises a CDR1 sequence identical to the amino acid sequence of SEQ ID NO:38, a CDR2 sequence identical to the amino acid sequence of SEQ ID NO:39, and a CDR3 sequence identical to the amino acid sequence of SEQ ID NO:40.

39. The antigen-binding site according to claim 37 or 38 having a K_{D} of between 6-600 nM, as measured by surface plasmon resonance.

40. A protein comprising:
an antigen-binding site according to any one of claims 25-39, wherein the antigen-binding site binds to NKG2D; and an additional antigen-binding site.

41. The protein according to claim 40, wherein the additional antigen-binding site binds to a tumor-associated antigen.

42. The protein according to claim 41, wherein the tumor-associated antigen is selected from the group consisting of CD33, HER2, EpCAM, CD2, CD19, CD20, CD30, CD38, CD40, CD52, CD70, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, and PD1.

43. The protein according to any one of claims 40-42, wherein the antigen-binding site binding NKG2D comprises a first antibody heavy chain variable domain, and the additional antigen-binding site comprises a second antibody heavy chain variable domain; and
wherein the first antibody heavy chain variable domain is present on a first polypeptide further comprising a first antibody constant region, and the second antibody heavy chain variable domain is present on a second polypeptide further comprising a second antibody constant region.

44. The protein according to claim 43, wherein the first antibody constant region and the second antibody constant region form a complex capable of binding CD16.

45. The protein according to claim 43 or 44, wherein the first antibody constant region and the second antibody constant region each comprise hinge, CH2, and CH3 domains.

46. The protein according to any one of claims 43-45, wherein the first antibody constant region and the second antibody constant region each further comprise a CH1 domain.

47. The protein according to any one of claims 43-46, wherein the amino acid sequences of the first antibody constant region and the second antibody constant region are each at least 90% identical to human IgG1 constant region.

48. The protein according to claim 47, wherein:
the amino acid sequence of the first antibody constant region differs from the amino acid sequence of an IgG1 constant region at Q347, Y349, L351, S354, E356, E357, K360, Q362, S364, T366, L368, K370, K392, T394, D399, S400, D401, F405, Y407, K409, T411, or K439, or any combination thereof; and
the amino acid sequence of the second antibody constant region differs from the amino acid sequence of an IgG1 constant region at Q347, Y349, L351, S354, E356, E357, S364, T366, L368, K370, N390, K392, T394, D399, D401, F405, Y407, K409, T411, or K439, or any combination thereof.

49. The protein according to claim 47, wherein the amino acid sequence of the first antibody constant region differs from the amino acid sequence of an IgG1 constant region at position T366, and wherein the amino acid sequence of the second antibody constant region differs from the amino acid sequence of an IgG1 constant region at T366, L368, or Y407, or any combination thereof.

50. The protein according to claim 47, wherein the amino acid sequence of the first antibody constant region differs from the amino acid sequence of an IgG1 constant region at T366, L368, or Y407, or any combination thereof, and wherein the amino acid sequence of the second antibody constant region differs from the amino acid sequence of an IgG1 constant region at position T366.

51. The protein according to claim 47, wherein the amino acid sequence of the first antibody constant region differs from the amino acid sequence of an IgG1 constant region at E357, K360, Q362, S364, L368, K370, T394, D401, F405, or T411, or any combination thereof, and wherein the amino acid sequence of the second antibody constant region differs from the amino acid sequence of an IgG1 constant region at Y349, E357, S364, L368, K370, T394, D401, F405, or T411, or any combination thereof.

52. The protein according to claim 47, wherein the amino acid sequence of the first antibody constant region differs from the amino acid sequence of an IgG1 constant region at Y349, E357, S364, L368, K370, T394, D401, F405, or T411, or any combination thereof, and wherein the amino acid sequence of the second antibody constant region differs from the amino acid sequence of an IgG1 constant region at E357, K360, Q362, S364, L368, K370, T394, D401, F405, or T411, or any combination thereof.

53. The protein according to claim 47, wherein the amino acid sequence of the first antibody constant region differs from the amino acid sequence of an IgG1 constant region at L351, D399,S400, or Y407, or any combination thereof, and wherein the amino acid sequence of the second antibody constant region differs from the amino acid sequence of an IgG1 constant region at T366, N390, K392, K409, or T411, or any combination thereof.

54. The protein according to claim 47, wherein the amino acid sequence of the a first antibody constant region differs from the amino acid sequence of an IgG1 constant region at T366, N390, K392, K409, or T411, or any combination thereof, and wherein the amino acid sequence of the second antibody constant region differs from the amino acid sequence of an IgG1 constant region at L351, D399, S400, or Y407, or any combination thereof.

55. The protein according to claim 47, wherein the amino acid sequence of the first antibody constant region differs from the amino acid sequence of an IgG1 constant region at Q347, Y349,K360, or K409, or any combination thereof, and wherein the amino acid sequence of the second antibody constant region differs from the amino acid sequence of an IgG1 constant region at Q347, E357, D399, or F405, or any combination thereof.

56. The protein according to claim 47, wherein the amino acid sequence of the first antibody constant region differs from the amino acid sequence of an IgG1 constant region at Q347, E357,D399, or F405, or any combination thereof, and wherein the amino acid sequence of the second antibody constant region differs from the amino acid sequence of an IgG1 constant region at Y349, K360, Q347, or K409, or any combination thereof.

57. The protein according to claim 47, wherein the amino acid sequence of the first antibody constant region differs from the amino acid sequence of an IgG1 constant region at K370, K392, K409, or K439, or any combination thereof, and wherein the amino acid sequence of the second antibody constant region differs from the amino acid sequence of an IgG1 constant region at D356, E357, or D399, or any combination thereof.

58. The protein according to claim 47, wherein the amino acid sequence of the first antibody constant region differs from the amino acid sequence of an IgG1 constant region at D356, E357,or D399, or any combination thereof, and wherein the amino acid sequence of the second antibody constant region differs from the amino acid sequence of an IgG1 constant region at K370, K392, K409, or K439, or any combination thereof.

59. The protein according to claim 47, wherein the amino acid sequence of the first antibody constant region differs from the amino acid sequence of an IgG1 constant region at L351, E356,T366, or D399, or any combination thereof, and wherein the amino acid sequence of the second antibody constant region differs from the amino acid sequence of an IgG1 constant region at Y349, L351, L368, K392, or K409, or any combination thereof.

60. The protein according to claim 47, wherein the amino acid sequence of the first antibody constant region differs from the amino acid sequence of an IgG1 constant region at Y349,L351, L368, K392, or K409, or any combination thereof, and wherein the amino acid sequence of the second antibody constant region differs from the amino acid sequence of an IgG1 constant region at L351, E356, T366, or D399, or any combination thereof.

61. The protein according to claim 47, wherein the amino acid sequence of the first antibody constant region differs from the amino acid sequence of an IgG1 constant region by an S354C substitution and wherein the amino acid sequence of the second antibody constant region differs from the amino acid sequence of an IgG1 constant region by a Y349C substitution.

62. The protein according to claim 47, wherein the amino acid sequence of the first antibody constant region differs from the amino acid sequence of an IgG1 constant region by a Y349C substitution and wherein the amino acid sequence of the second antibody constant region differs from the amino acid sequence of an IgG1 constant region by an S354C substitution.

63. The protein according to claim 47, wherein the amino acid sequence of the first antibody constant region differs from the amino acid sequence of an IgG1 constant region by K360E and K409W substitutions and wherein the amino acid sequence of the second antibody constant region differs from the amino acid sequence of an IgG1 constant region by 0347R, D399V and F405T substitutions.

64. The protein according to claim 47, wherein the amino acid sequence of the first antibody constant region differs from the amino acid sequence of an IgG1 constant region by 0347R, D399V and F405T substitutions and wherein the amino acid sequence of the second antibody constant region differs from the amino acid sequence of an IgG1 constant region by K360E and K409W substitutions.

65. The protein according to claim 47, wherein the amino acid sequence of the first antibody constant region differs from the amino acid sequence of an IgG1 constant region by a T366W substitutions and wherein the amino acid sequence of the second antibody constant region differs from the amino acid sequence of an IgG1 constant region by T366S, T368A, and Y407V substitutions.

66. The protein according to claim 47, wherein the amino acid sequence of the first antibody constant region differs from the amino acid sequence of an IgG1 constant region by T366S, T368A, and Y407V substitutions and wherein the amino acid sequence of the second antibody constant region differs from the amino acid sequence of an IgG1 constant region by a T366W substitution.

67. The protein according to claim 47, wherein the amino acid sequence of the first antibody constant region differs from the amino acid sequence of an IgG1 constant region by T350V, L351Y, F405A, and Y407V substitutions and wherein the amino acid sequence of the second antibody constant region differs from the amino acid sequence of an IgG1 constant region by T350V, T366L, K392L, and T394W substitutions.

68. The protein according to claim 47, wherein the amino acid sequence of the first antibody constant region differs from the amino acid sequence of an IgG1 constant region by T350V, T366L, K392L, and T394W substitutions and wherein the amino acid sequence of the second antibody constant region differs from the amino acid sequence of an IgG1 constant region by T350V, L351Y, F405A, and Y407V substitutions.

69. The protein according to any one of claims 40-42, wherein the protein further comprises an antigen-binding site capable of binding CD16.

70. A protein comprising an antigen-binding site that competes for binding to human and/or cynomolgus NKG2D with an antibody comprising a heavy chain variable region having the amino acid sequence of SEQ ID NO:7 and a light chain variable region having the amino acid sequence of SEQ ID NO:8.

71. A protein comprising an antigen-binding site that competes for binding to human and/or cynomolgus NKG2D with an antibody comprising a heavy chain variable region having the amino acid sequence of SEQ ID NO:85 and a light chain variable region having the amino acid sequence of SEQ ID NO:8.

72. A protein comprising an antigen-binding site that competes for binding to human and/or cynomolgus NKG2D with an antibody comprising a heavy chain variable region having the amino acid sequence of SEQ ID NO: 1 and a light chain variable region having the amino acid sequence of SEQ ID NO:2.

73. A protein comprising an antigen-binding site that competes for binding to human and/or cynomolgus NKG2D with an antibody comprising a heavy chain variable region having the amino acid sequence of SEQ ID NO:3 and a light chain variable region having the amino acid sequence of SEQ ID NO:4.

74. A protein comprising an antigen-binding site that competes for binding to human and/or cynomolgus NKG2D with an antibody comprising a heavy chain variable region having the amino acid sequence of SEQ ID NO:5 and a light chain variable region having the amino acid sequence of SEQ ID NO:6.

75. A protein comprising an antigen-binding site that competes for binding to human and/or cynomolgus NKG2D with an antibody comprising a heavy chain variable region having the amino acid sequence of SEQ ID NO:9 and a light chain variable region having the amino acid sequence of SEQ ID NO:20.

76. A formulation comprising the protein according to any one of claims 40-75 and a pharmaceutically acceptable carrier.

77. A cell comprising one or more nucleic acids encoding the protein according to any one of claims 40-75.

78. A method of enhancing tumor cell death, the method comprising exposing a tumor and natural killer cells to the protein according to any one of claims 40-75.

79. A method of treating cancer, wherein the method comprises administering the protein according to any one of claims 40-75 or the formulation according to claim 76 to a patient.

80. The method according to claim 79, wherein the cancer is selected from the group consisting of acute myeloid leukemia, acute myelomonocytic leukemia, B cell lymphoma, bladder cancer, breast cancer, colorectal cancer, diffuse large B cell lymphoma esophageal cancer, Ewing's sarcoma, follicular lymphoma, gastric cancer, gastrointestinal cancer, gastrointestinal stromal tumors, glioblastoma, head and neck cancer, melanoma, mesothelioma, multiple myeloma, myelodysplastic syndrome, renal cell carcinoma, neuroblastoma, non-small cell lung cancer, neuroendocrine tumors, ovarian cancer, and pancreatic cancer, prostate cancer, sarcomas, small cell lung cancer, T cell lymphoma, testis cancer, thymic carcinoma, thyroid cancer, urothelial cancer, cancers infiltrated by myeloid-derived suppressor cells, cancers with extracellular matrix deposition, cancers with high levels of reactive stroma, and cancers with neoangiogenesis.


SEQUENCE LISTING (86 sequences)

SEQ ID NO: 1 - protein, 126 aa, synthetic construct
  REGION 1..126: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 2 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 3 - protein, 124 aa, synthetic construct
  REGION 1..124: note = Description of Artificial Sequence: Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 4 - protein, 107 aa, synthetic construct
  REGION 1..107: note = Description of Artificial Sequence: Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 5 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 6 - protein, 107 aa, synthetic construct
  REGION 1..107: note = Description of Artificial Sequence: Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 7 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 8 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 9 - protein, 125 aa, synthetic construct
  REGION 1..125: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 10 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 11 - protein, 9 aa, synthetic construct
  REGION 1..9: note = Description of Artificial Sequence: Synthetic peptide
YTFTSYYMH

SEQ ID NO: 12 - protein, 17 aa, synthetic construct
  REGION 1..17: note = Description of Artificial Sequence: Synthetic peptide
IINPSGGSTSYAQKFQG

SEQ ID NO: 13 - protein, 19 aa, synthetic construct
  REGION 1..19: note = Description of Artificial Sequence: Synthetic peptide
ARGAPNYGDTTHDYYYMDV

SEQ ID NO: 14 - protein, 11 aa, synthetic construct
  REGION 1..11: note = Description of Artificial Sequence: Synthetic peptide
RASQSVSSNLA

SEQ ID NO: 15 - protein, 7 aa, synthetic construct
  REGION 1..7: note = Description of Artificial Sequence: Synthetic peptide
GASTRAT

SEQ ID NO: 16 - protein, 9 aa, synthetic construct
  REGION 1..9: note = Description of Artificial Sequence: Synthetic peptide
QQYDDWPFT

SEQ ID NO: 17 - protein, 9 aa, synthetic construct
  REGION 1..9: note = Description of Artificial Sequence: Synthetic peptide
YTFTGYYMH

SEQ ID NO: 18 - protein, 17 aa, synthetic construct
  REGION 1..17: note = Description of Artificial Sequence: Synthetic peptide
WINPNSGGTNYAQKFQG

SEQ ID NO: 19 - protein, 17 aa, synthetic construct
  REGION 1..17: note = Description of Artificial Sequence: Synthetic peptide
ARDTGEYYDTDDHGMDV

SEQ ID NO: 20 - protein, 11 aa, synthetic construct
  REGION 1..11: note = Description of Artificial Sequence: Synthetic peptide
RASQSVSSNLA

SEQ ID NO: 21 - protein, 7 aa, synthetic construct
  REGION 1..7: note = Description of Artificial Sequence: Synthetic peptide
GASTRAT

SEQ ID NO: 22 - protein, 9 aa, synthetic construct
  REGION 1..9: note = Description of Artificial Sequence: Synthetic peptide
QQDDYWPPT

SEQ ID NO: 23 - protein, 9 aa, synthetic construct
  REGION 1..9: note = Description of Artificial Sequence: Synthetic peptide
FTFSSYAMS

SEQ ID NO: 24 - protein, 17 aa, synthetic construct
  REGION 1..17: note = Description of Artificial Sequence: Synthetic peptide
AISGSGGSTYYADSVKG

SEQ ID NO: 25 - protein, 14 aa, synthetic construct
  REGION 1..14: note = Description of Artificial Sequence: Synthetic peptide
AKDGGYYDSGAGDY

SEQ ID NO: 26 - protein, 11 aa, synthetic construct
  REGION 1..11: note = Description of Artificial Sequence: Synthetic peptide
RASQGIDSWLA

SEQ ID NO: 27 - protein, 7 aa, synthetic construct
  REGION 1..7: note = Description of Artificial Sequence: Synthetic peptide
AASSLQS

SEQ ID NO: 28 - protein, 9 aa, synthetic construct
  REGION 1..9: note = Description of Artificial Sequence: Synthetic peptide
QQGVSYPRT

SEQ ID NO: 29 - protein, 9 aa, synthetic construct
  REGION 1..9: note = Description of Artificial Sequence: Synthetic peptide
FTFSSYSMN

SEQ ID NO: 30 - protein, 17 aa, synthetic construct
  REGION 1..17: note = Description of Artificial Sequence: Synthetic peptide
SISSSSSYIYYADSVKG

SEQ ID NO: 31 - protein, 15 aa, synthetic construct
  REGION 1..15: note = Description of Artificial Sequence: Synthetic peptide
ARGAPMGAAAGWFDP

SEQ ID NO: 32 - protein, 11 aa, synthetic construct
  REGION 1..11: note = Description of Artificial Sequence: Synthetic peptide
RASQGISSWLA

SEQ ID NO: 33 - protein, 7 aa, synthetic construct
  REGION 1..7: note = Description of Artificial Sequence: Synthetic peptide
AASSLQS

SEQ ID NO: 34 - protein, 9 aa, synthetic construct
  REGION 1..9: note = Description of Artificial Sequence: Synthetic peptide
QQGVSFPRT

SEQ ID NO: 35 - protein, 9 aa, synthetic construct
  REGION 1..9: note = Description of Artificial Sequence: Synthetic peptide
YTFTSYYMH

SEQ ID NO: 36 - protein, 17 aa, synthetic construct
  REGION 1..17: note = Description of Artificial Sequence: Synthetic peptide
IINPSGGSTSYAQKFQG

SEQ ID NO: 37 - protein, 18 aa, synthetic construct
  REGION 1..18: note = Description of Artificial Sequence: Synthetic peptide
AREGAGFAYGMDYYYMDV

SEQ ID NO: 38 - protein, 11 aa, synthetic construct
  REGION 1..11: note = Description of Artificial Sequence: Synthetic peptide
RASQSVSSYLA

SEQ ID NO: 39 - protein, 7 aa, synthetic construct
  REGION 1..7: note = Description of Artificial Sequence: Synthetic peptide
DASNRAT

SEQ ID NO: 40 - protein, 9 aa, synthetic construct
  REGION 1..9: note = Description of Artificial Sequence: Synthetic peptide
QQSDNWPFT

SEQ ID NO: 41 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 42 - protein, 122 aa, synthetic construct
  REGION 1..122: note = Description of Artificial Sequence: Synthetic polypeptide
  VARIANT 102: note = Met, Leu, Ile, Val, Gln or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 43 - protein, 15 aa, synthetic construct
  REGION 1..15: note = Description of Artificial Sequence: Synthetic peptide
  VARIANT 6: note = Met, Leu, Ile, Val, Gln or Phe
ARGAPXGAAAGWFDP

SEQ ID NO: 44 - protein, 13 aa, synthetic construct
  REGION 1..13: note = Description of Artificial Sequence: Synthetic peptide
  VARIANT 4: note = Met, Leu, Ile, Val, Gln or Phe
GAPXGAAAGWFDP

SEQ ID NO: 45 - protein, 5 aa, synthetic construct
  REGION 1..5: note = Description of Artificial Sequence: Synthetic peptide
SYYMH

SEQ ID NO: 46 - protein, 5 aa, synthetic construct
  REGION 1..5: note = Description of Artificial Sequence: Synthetic peptide
GYYMH

SEQ ID NO: 47 - protein, 5 aa, synthetic construct
  REGION 1..5: note = Description of Artificial Sequence: Synthetic peptide
SYAMS

SEQ ID NO: 48 - protein, 5 aa, synthetic construct
  REGION 1..5: note = Description of Artificial Sequence: Synthetic peptide
SYSMN

SEQ ID NO: 49 - protein, 120 aa, synthetic construct
  REGION 1..120: note = Description of Artificial Sequence: Synthetic polypeptide
QVQLVQSGAEVKKPGASVKVSCKASGYTFTDYVVHWVRQAPGQGLEWMGYINPYNDGTKYNEKFKGRVTMTRDTSISTAYMELSRLRSDDTAVYYCARDYRYEVYGMDYWGQGTLVTVSS

SEQ ID NO: 50 - protein, 106 aa, synthetic construct
  REGION 1..106: note = Description of Artificial Sequence: Synthetic polypeptide
DIVLTQSPASLAVSPGQRATITCTASSSVNYIHWYQQKPGQPPKLLIYDTSKVASGVPARFSGSGSGTDFTLTINPVEANDTANYYCQQWRSYPLTFGQGTKLEIK

SEQ ID NO: 51 - protein, 117 aa, synthetic construct
  REGION 1..117: note = Description of Artificial Sequence: Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 52 - protein, 9 aa, synthetic construct
  REGION 1..9: note = Description of Artificial Sequence: Synthetic peptide
GSFSGYYWS

SEQ ID NO: 53 - protein, 16 aa, synthetic construct
  REGION 1..16: note = Description of Artificial Sequence: Synthetic peptide
EIDHSGSTNYNPSLKS

SEQ ID NO: 54 - protein, 11 aa, synthetic construct
  REGION 1..11: note = Description of Artificial Sequence: Synthetic peptide
ARARGPWSFDP

SEQ ID NO: 55 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 56 - protein, 11 aa, synthetic construct
  REGION 1..11: note = Description of Artificial Sequence: Synthetic peptide
RASQSISSWLA

SEQ ID NO: 57 - protein, 7 aa, synthetic construct
  REGION 1..7: note = Description of Artificial Sequence: Synthetic peptide
KASSLES

SEQ ID NO: 58 - protein, 8 aa, synthetic construct
  REGION 1..8: note = Description of Artificial Sequence: Synthetic peptide
QQYDTFIT

SEQ ID NO: 59 - protein, 115 aa, synthetic construct
  REGION 1..115: note = Description of Artificial Sequence: Synthetic polypeptide
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 60 - protein, 5 aa, synthetic construct
  REGION 1..5: note = Description of Artificial Sequence: Synthetic peptide
SYYWS

SEQ ID NO: 61 - protein, 16 aa, synthetic construct
  REGION 1..16: note = Description of Artificial Sequence: Synthetic peptide
HISYSGSANYNPSLKS

SEQ ID NO: 62 - protein, 7 aa, synthetic construct
  REGION 1..7: note = Description of Artificial Sequence: Synthetic peptide
WDDAFNI

SEQ ID NO: 63 - protein, 108 aa, synthetic construct
  REGION 1..108: note = Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGGGTKVEIK

SEQ ID NO: 64 - protein, 12 aa, synthetic construct
  REGION 1..12: note = Description of Artificial Sequence: Synthetic peptide
RASQSVSSSYLA

SEQ ID NO: 65 - protein, 7 aa, synthetic construct
  REGION 1..7: note = Description of Artificial Sequence: Synthetic peptide
GASSRAT

SEQ ID NO: 66 - protein, 9 aa, synthetic construct
  REGION 1..9: note = Description of Artificial Sequence: Synthetic peptide
QQYGSSPWT

SEQ ID NO: 67 - protein, 193 aa, Homo sapiens
RRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSG

SEQ ID NO: 68 - protein, 17 aa, synthetic construct
  REGION 1..17: note = Description of Artificial Sequence: Synthetic peptide
GAPNYGDTTHDYYYMDV

SEQ ID NO: 69 - protein, 15 aa, synthetic construct
  REGION 1..15: note = Description of Artificial Sequence: Synthetic peptide
DTGEYYDTDDHGMDV

SEQ ID NO: 70 - protein, 12 aa, synthetic construct
  REGION 1..12: note = Description of Artificial Sequence: Synthetic peptide
DGGYYDSGAGDY

SEQ ID NO: 71 - protein, 13 aa, synthetic construct
  REGION 1..13: note = Description of Artificial Sequence: Synthetic peptide
GAPMGAAAGWFDP

SEQ ID NO: 72 - protein, 16 aa, synthetic construct
  REGION 1..16: note = Description of Artificial Sequence: Synthetic peptide
EGAGFAYGMDYYYMDV

SEQ ID NO: 73 - protein, 15 aa, synthetic construct
  REGION 1..15: note = Description of Artificial Sequence: Synthetic peptide
ARGAPQGAAAGWFDP

SEQ ID NO: 74 - protein, 13 aa, synthetic construct
  REGION 1..13: note = Description of Artificial Sequence: Synthetic peptide
GAPQGAAAGWFDP

SEQ ID NO: 75 - protein, 15 aa, synthetic construct
  REGION 1..15: note = Description of Artificial Sequence: Synthetic peptide
ARGAPLGAAAGWFDP

SEQ ID NO: 76 - protein, 13 aa, synthetic construct
  REGION 1..13: note = Description of Artificial Sequence: Synthetic peptide
GAPLGAAAGWFDP

SEQ ID NO: 77 - protein, 15 aa, synthetic construct
  REGION 1..15: note = Description of Artificial Sequence: Synthetic peptide
ARGAPIGAAAGWFDP

SEQ ID NO: 78 - protein, 13 aa, synthetic construct
  REGION 1..13: note = Description of Artificial Sequence: Synthetic peptide
GAPIGAAAGWFDP

SEQ ID NO: 79 - protein, 15 aa, synthetic construct
  REGION 1..15: note = Description of Artificial Sequence: Synthetic peptide
ARGAPFGAAAGWFDP

SEQ ID NO: 80 - protein, 13 aa, synthetic construct
  REGION 1..13: note = Description of Artificial Sequence: Synthetic peptide
GAPFGAAAGWFDP

SEQ ID NO: 81 - protein, 15 aa, synthetic construct
  REGION 1..15: note = Description of Artificial Sequence: Synthetic peptide
ARGAPVGAAAGWFDP

SEQ ID NO: 82 - protein, 13 aa, synthetic construct
  REGION 1..13: note = Description of Artificial Sequence: Synthetic peptide
GAPVGAAAGWFDP

SEQ ID NO: 83 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 84 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 85 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 86 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS